(19) United States
(12) Patent Application Publication
(10) Pub. No.: US 2024/0212801 A1
(43) Pub. Date: Jun. 27, 2024

(54) DYNAMIC PRESENTATION OF CROSS-FEATURE CORRELATION INSIGHTS FOR CONTINUOUS ANALYTE DATA

(71) Applicant: Dexcom, Inc., San Diego, CA (US)

(72) Inventors: Stacey Lynne FISCHER, San Diego, CA (US); Afshan A. KLEINHANZL, San Diego, CA (US); Sonya Ann FRANGOPOULOS, San Diego, CA (US); Nikita SRIDHAR, San Diego, CA (US); Nicholas Alexander FRATI, San Diego, CA (US); Colton Alfredo Tanner PLAZA, San Diego, CA (US); Kerry Roller JOHNSON, San Diego, CA (US)

(21) Appl. No.: 18/545,956

(22) Filed: Dec. 19, 2023

Related U.S. Application Data
(60) Provisional application No. 63/476,921, filed on Dec. 22, 2022.

(51) Int. Cl. G16H 10/20; A61B 5/145; G16H 20/00; G16H 50/30

(52) U.S. Cl. CPC G16H 10/20; A61B 5/14532; G16H 20/00; G16H 50/30

(57) ABSTRACT

Systems, devices, and methods for dynamic determination and presentation of cross-feature correlation insights are provided. In one embodiment, a non-transitory computer readable storage medium storing a program is provided, the program comprising instructions that, when executed by at least one processor of a computing device, cause the at least one processor to perform operations including identifying at least one analyte feature using an analyte feature selection user interface (UI); identifying at least one correlative feature using a correlative feature selection UI; determining an analyte feature trend for the at least one analyte feature; determining a correlative feature trend for the at least one correlative feature; determining at least one cross-feature correlation insight based on the analyte feature and the correlative feature trends; determining a correlation magnitude profile for the at least one cross-feature correlation; and displaying the at least one cross-feature correlation insight using a at least one insight UI.

### CROSS-REFERENCE TO RELATED APPLICATIONS

This application claims priority to and benefit of U.S. Provisional Application No. 63/476,921, filed Dec. 22, 2022, which is assigned to the assignee hereof and hereby expressly incorporated by reference herein in its entirety as if fully set forth below and for all applicable purposes.

### FIELD

### Background

This application generally relates to medical devices (e.g., analyte sensors), and more specifically to systems, devices, and methods for providing insight for improving patients' health outcomes.

### DESCRIPTION OF THE RELATED TECHNOLOGY

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps may also be utilized to receive insulin. Diet and exercise also affect blood glucose levels.

Diabetes conditions may be referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

This background is provided to introduce a brief context for the summary and detailed description that follow. This background is not intended to be an aid in determining the scope of the claimed subject matter nor be viewed as limiting the claimed subject matter to implementations that solve any or all of the disadvantages or problems presented above.

### BRIEF SUMMARY

The various embodiments of the present systems, devices, and methods for dynamic determination and presentation of cross-feature correlation insights for improving patients' health outcomes comprise several features, no single one of which is solely responsible for their desirable attributes. Without limiting the scope of the present embodiments, their more prominent features will now be discussed below. After considering this discussion, and particularly after reading the section entitled “Detailed Description,” one will understand how the features of the present embodiments provide the advantages described herein.

In a first aspect, a non-transitory computer readable storage medium is provided, the non-transitory computer readable storage medium storing a program comprising instructions that, when executed by at least one processor of a computing device, cause the at least one processor to perform operations including: identifying at least one analyte feature using an analyte feature selection user interface (UI); identifying at least one correlative feature using a correlative feature selection UI; determining an analyte feature trend for the at least one analyte feature, wherein the analyte feature trend corresponds to fluctuations of the at least one analyte feature over a period of time; determining a correlative feature trend for the at least one correlative feature, wherein the correlative feature trend corresponds to fluctuations of the at least one correlative feature over the period of time; determining at least one cross-feature correlation insight based on the analyte feature trend and the correlative feature trend, wherein the at least one cross-feature correlation insight is based on a correlation between the at least one analyte feature and the at least one correlative feature; determining a correlation magnitude profile for the at least one cross-feature correlation, wherein the correlation magnitude profile includes correlation magnitudes for the at least one cross-feature correlation insight over a correlation period; and displaying the at least one cross-feature correlation insight using at least one insight UI.

In an embodiment of the first aspect, the at least one analyte feature is identified by displaying the analyte feature selection UI and receiving a user input selecting the at least one analyte feature.

In another embodiment of the first aspect, the at least one corresponding feature is identified by displaying the corresponding feature selection UI and receiving a user input selecting the at least one corresponding feature.

In another embodiment of the first aspect, the at least one cross-feature correlation insight is displayed using an insight flagging UI, and wherein the insight flagging UI provides UI elements for a user to flag a cross-feature correlation insight.

In another embodiment of the first aspect, the at least one cross-feature correlation insight is displayed using a cross-temporal insight interaction UI, and wherein the cross-temporal insight interaction UI: provides UI elements for the user to select correlation periods; and displays the correlation magnitude profile for the flagged cross-feature correlation insight with respect to the user-selected correlation periods.

In another embodiment of the first aspect, the operations further comprise: receiving a user selected correlation period using the cross-temporal insight interaction UI; updating the correlation magnitude for the flagged cross-feature correction insight; and displaying the updated correlation magnitude profile for the flagged cross-feature correlation insight using the interaction UI.

In another embodiment of the first aspect, the at least one cross-feature correlation insight is displayed using a set of flagged insight engagement UIs, and wherein the set of insight engagement UIs: provide UI elements for the user to edit the flagged cross-feature correlation insight; and assign metadata fields to the flagged cross-feature correlation insight.

In another embodiment of the first aspect, the at least one cross-feature correlation insight is displayed using a set of content engagement UIs, and wherein the set of content engagement UIs: display educational content curated based on the flagged cross-feature insight; and provide UI elements for the user to engage with the educational content.

In a second aspect, a method for dynamic determination and presentation of cross-feature correlation insights is provided, the method comprising: identifying at least one analyte feature using an analyte feature selection user interface (UI); identifying at least one correlative feature using a correlative feature selection UI; determining an analyte feature trend for the at least one analyte feature, wherein the analyte feature trend corresponds to fluctuations of the at least one analyte feature over a period of time; determining a correlative feature trend for the at least one correlative feature, wherein the correlative feature trend corresponds to fluctuations of the at least one correlative feature over the period of time; determining at least one cross-feature correlation insight based on the analyte feature trend and the correlative feature trend, wherein the at least one cross-feature correlation insight is based on a correlation between the at least one analyte feature and the at least one correlative feature; determining a correlation magnitude profile for the at least one cross-feature correlation, wherein the correlation magnitude profile includes correlation magnitudes for the at least one cross-feature correlation insight over a correlation period; and displaying the at least one cross-feature correlation insight using at least one insight UI.

In an embodiment of the second aspect, the at least one analyte feature is identified by displaying the analyte feature selection UI and receiving a user input selecting the at least one analyte feature.

In another embodiment of the second aspect, the at least one corresponding feature is identified by displaying the corresponding feature selection UI and receiving a user input selecting the at least one corresponding feature.

In another embodiment of the second aspect, the at least one cross-feature correlation insight is displayed using an insight flagging UI, and wherein the insight flagging UI provides UI elements for a user to flag a cross-feature correlation insight.

In another embodiment of the second aspect, the at least one cross-feature correlation insight is displayed using a cross-temporal insight interaction UI, and wherein the cross-temporal insight interaction UI: provides UI elements for the user to select correlation periods; and displays the correlation magnitude profile for the flagged cross-feature correlation insight with respect to the user-selected correlation periods.

In another embodiment of the second aspect, the at least one cross-feature correlation insight is displayed using a set of flagged insight engagement UIs, and wherein the set of insight engagement UIs: provide UI elements for the user to edit the flagged cross-feature correlation insight; and assign metadata fields to the flagged cross-feature correlation insight.

In another embodiment of the second aspect, the at least one cross-feature correlation insight is displayed using a set of content engagement UIs, and wherein the set of content engagement UIs: display educational content curated based on the flagged cross-feature insight; and provide UI elements for the user to engage with the educational content.

In a third aspect, a computing device for dynamic determination and presentation of cross-feature correlation insights is provided, the computing device comprising: a network interface; a memory comprising executable instructions; a processor in data communication with the memory and configured to execute the instructions to: identify at least one analyte feature using an analyte feature selection user interface (UI); identify at least one correlative feature using a correlative feature selection UI; determine an analyte feature trend for the at least one analyte feature, wherein the analyte feature trend corresponds to fluctuations of the at least one analyte feature over a period of time; determine a correlative feature trend for the at least one correlative feature, wherein the correlative feature trend corresponds to fluctuations of the at least one correlative feature over the period of time; determine at least one cross-feature correlation insight based on the analyte feature trend and the correlative feature trend, wherein the at least one cross-feature correlation insight is based on a correlation between the at least one analyte feature and the at least one correlative feature; determine a correlation magnitude profile for the at least one cross-feature correlation, wherein the correlation magnitude profile includes correlation magnitudes for the at least one cross-feature correlation insight over a correlation period; and display the at least one cross-feature correlation insight using at least one insight UI.

In an embodiment of the third aspect, the at least one analyte feature is identified by displaying the analyte feature selection UI and receiving a user input selecting the at least one analyte feature.

In another embodiment of the third aspect, the at least one corresponding feature is identified by displaying the corresponding feature selection UI and receiving a user input selecting the at least one corresponding feature.

In another embodiment of the third aspect, the at least one cross-feature correlation insight is displayed using an insight flagging UI, and wherein the insight flagging UI provides UI elements for a user to flag a cross-feature correlation insight.

In another embodiment of the third aspect, the at least one cross-feature correlation insight is displayed using a cross-temporal insight interaction UI, and wherein the cross-temporal insight interaction UI: provides UI elements for the user to select correlation periods; and displays the correlation magnitude profile for the flagged cross-feature correlation insight with respect to the user-selected correlation periods.

### BRIEF DESCRIPTION OF THE DRAWINGS

FIG. 1A illustrates an example health monitoring and support system, in accordance with certain embodiments of the disclosure.

FIG. 1B illustrates a continuous analyte monitoring system, in accordance with certain embodiments of the disclosure.

FIG. 2 illustrates example inputs and example metrics that are generated based on the inputs, in accordance with certain embodiments of the disclosure.

FIG. 3 is a flow diagram illustrating exemplary user interfaces (UIs) for use with dynamic determination and presentation of cross-feature correlation insights, in accordance with certain embodiments of the disclosure.

FIG. 4 is a flow diagram illustrating an exemplary process for dynamic determination and presentation of cross-feature correlation insights, in accordance with certain embodiments of the disclosure.

FIG. 5 illustrates an analyte feature selection UI for identifying at least one analyte feature used in the process of FIG. 4, in accordance with certain embodiments of the disclosure.

FIG. 6 illustrates a correlative feature selection UI for identifying at least one correlative feature used in the process of FIG. 4, in accordance with certain embodiments of the disclosure.

FIG. 7 illustrates an insight flagging UI for presenting cross-feature correlation insights as part of the process of FIG. 4, in accordance with certain embodiments of the disclosure.

FIG. 8 illustrates a cross-temporal insight interaction UI for presenting cross-feature correlation insights as part of the process of FIG. 4, in accordance with certain embodiments of the disclosure.

FIG. 9 illustrates a flagged insight engagement UI for presenting cross-feature correlation insights as part of the process of FIG. 4, in accordance with certain embodiments of the disclosure.

FIG. 10 illustrates another flagged insight engagement UI for assigning metadata for a flagged cross-feature correlation insight of FIG. 9, in accordance with certain embodiments of the disclosure.

FIG. 11 illustrates another flagged insight engagement UI for modifying a flagged cross-feature correlation insight of FIG. 9, in accordance with certain embodiments of the disclosure.

FIG. 12 illustrates a content engagement UI for presenting cross-feature correlation insights determined as part of the process of FIG. 4, in accordance with certain embodiments of the disclosure.

FIG. 13 illustrates another content engagement UI for engaging with education content of FIG. 12, in accordance with certain embodiments of the disclosure.

FIG. 14 is a block diagram depicting a computing device configured for dynamic determination and presentation of cross-feature correlation insights, in accordance with certain embodiments of the disclosure.

### DETAILED DESCRIPTION

Portable and/or wearable health monitoring devices (also referred to herein as “health monitoring devices”) and mobile health applications (also referred to herein as “applications”), have rapidly become renowned for their capabilities to support user-centered care. For example, management of diabetes can present complex challenges for patients, clinicians, and caregivers, as a confluence of many factors can impact a patient's glucose level and glucose trends. To assist patients with better managing this condition, health monitoring devices (e.g., sensors and other types of monitoring and diagnostic devices) as well as a variety of mobile health applications (e.g., diabetes intervention software applications) have been developed. The wide dissemination of health monitoring devices and the increase in the development and distribution of mobile health applications has improved health management, and more specifically chronic disease management, in the healthcare domain. In particular, the use of mobile health applications in conjunction with these health monitoring devices, represents a more scalable and potentially more cost effective alternative to traditional interventions, offering a means of improving health and chronic disease management by expanding the reach of healthcare services and improving users' access to health-related information and interventions.

Mobile health applications enable users to be much more involved in the users' own medical care by granting them access to and control over their health information. In particular, mobile health applications enable users to access, monitor, record, and update their health information regardless of physical constraints, such as time and location. In particular, a variety of intervention applications have been developed to deliver guidance that may assist patients, caregivers, healthcare providers, or other users in improving lifestyle or clinical/patient outcomes by meeting a variety of challenges, such as analyte control, exercise, and/or other health factors. For example, diabetes intervention applications may assist patients, caregivers, healthcare providers, or other users in overnight glucose control (e.g., reduce incidence of hypoglycemic events or hyperglycemic excursions), glucose control during and after meals (e.g., use historical information and trends to increase glycemic control), hyperglycemia corrections (e.g., increase time in target zone while avoiding hypoglycemic events from over-correction), and/or hypoglycemia treatments (e.g., address hypoglycemia while avoiding “rebound” hyperglycemia), to name a few.

Unfortunately, many health-related applications suffer from high user attrition rates resulting from failure to provide health management assistance features that enable users to holistically manage their health conditions (e.g., their Type 2 diabetes condition) by setting their lifestyle choices and treatment plans in accordance with their monitored health conditions. For example, many health-related applications fail to provide personalized health management insights based on observed correlations between various health trends of a user. As another example, many health-related applications fail to enable a user to better prepare for medical appointments based on her personal health journey or receive educational content that is curated based on her monitored health conditions.

Accordingly, certain embodiments described herein provide a software application configured to be executed on a computing device (e.g., a mobile device) in communication with an analyte monitoring system (e.g., a glucose sensor system, such as a continuous glucose sensor system that is used for continuous glucose monitoring), where the software application provides a variety of functionalities and user interface (UI) views for enabling a user to holistically manage their health conditions based on how sensor data (also be referred to herein as “analyte data”) reported by the analyte monitoring system correlates with other health indicators of the user. In particular, certain embodiments described herein detect “cross-feature correlation insights” between one or more analyte features of the user and one or more correlative features of the user.

A cross-feature correlation insight is a determined correlation between an analyte feature of a user and a correlative feature of the user. An analyte feature of the user is a feature that is determined based on the sensor data reported by an analyte monitoring system, while a correlative feature is a feature whose potential correlation with an analyte feature is analyzed. Examples of analyte features include a time-in-range feature, an A1C analyte feature, a glucose high feature, and a glucose low feature. Examples of correlative features include a sleep level feature, a medication intake feature, a food intake feature, exercise level feature, stress level feature, heart rate feature, etc. An example of a cross-feature correlation insight is a determined correlation between a time-in-range feature and a sleep level feature for a user, such as a detected observation that a 20% increase in the amount of sleep by the user has been correlated with a 40% increase in the user's weekly time-in-range.

In some embodiments, after the software application detects a cross-feature correlation insight, the software application presents the insight using various UIs that, for example, enable the user to flag the insight for follow-up discussion in an upcoming medical appointment, engage with educational content curated based on the insight, and view how the magnitude of the underlying correlation changes as the “correlation period” for the insight is modified (e.g., as the period used for determining the correlation magnitude is changed from weekly to monthly).

In some embodiments, to enable the described functionalities herein, the software application uses: (i) feature selection UIs (e.g., an analyte feature selection UI and a correlative feature selection UI) that enable the user to select one or more analyte features and one or more correlative features for analysis (ii) an insight flagging UI that enables the user to flag a cross-feature correlation insight for discussion in a follow-up medical appointment, (iii) a cross-temporal insight interaction UI that enables the user to view correlation magnitude data for the cross-feature correlation insight across various correlation periods, (iv) a set of flagged insight engagement UIs that enables the user to edit cross-feature correlation insights that were previously flagged using the insight flagging UI and to assign various metadata fields to the flagged cross-feature correlation insights, and (v) a set of content engagement UIs that enable the user to engage with content data curated based on the cross-feature correlation insight. The insight flagging UI, cross-temporal insight interaction UI, the cross-temporal insight interaction UI, the set of flagged insight engagement UIs, and the set of content engagement UIs may be collectively referred to herein as “insight UIs.”

The systems, devices, and methods of the embodiments described herein can be used in conjunction with any type of analyte sensor for any measurable analyte. The term “analyte” as used herein refers without limitation to a substance or chemical constituent in the body or a biological sample. Further, the system, devices, and methods of the embodiment described herein may be used in conjunction with any health-related application that is provided to the user to improve the user's health. For example, a health-related application may help the user with treating a certain disease or just help with improving the health of a user who is not necessarily diagnosed with a disease.

Example System with a Cross-Feature Correlation Engine for Dynamic Determination and Presentation of Cross-Feature Correlation Insights

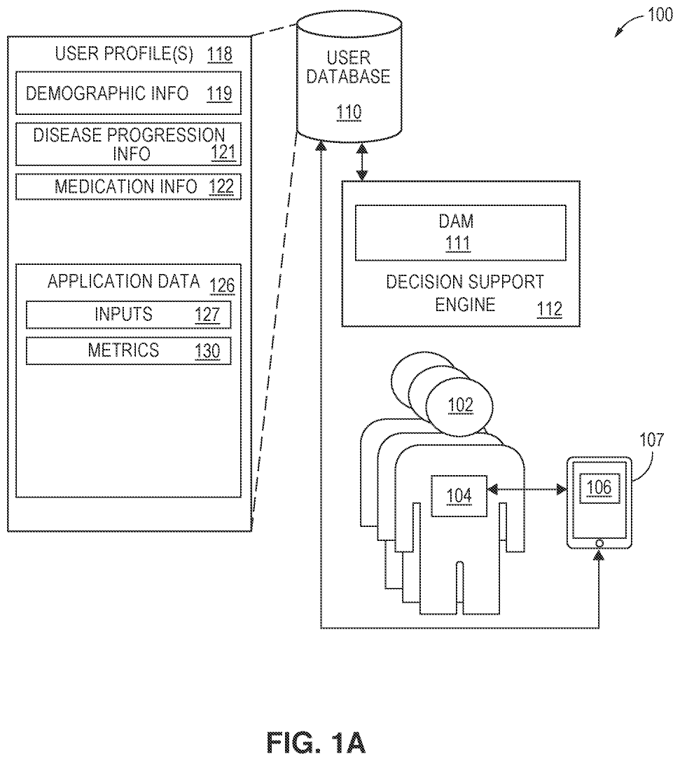

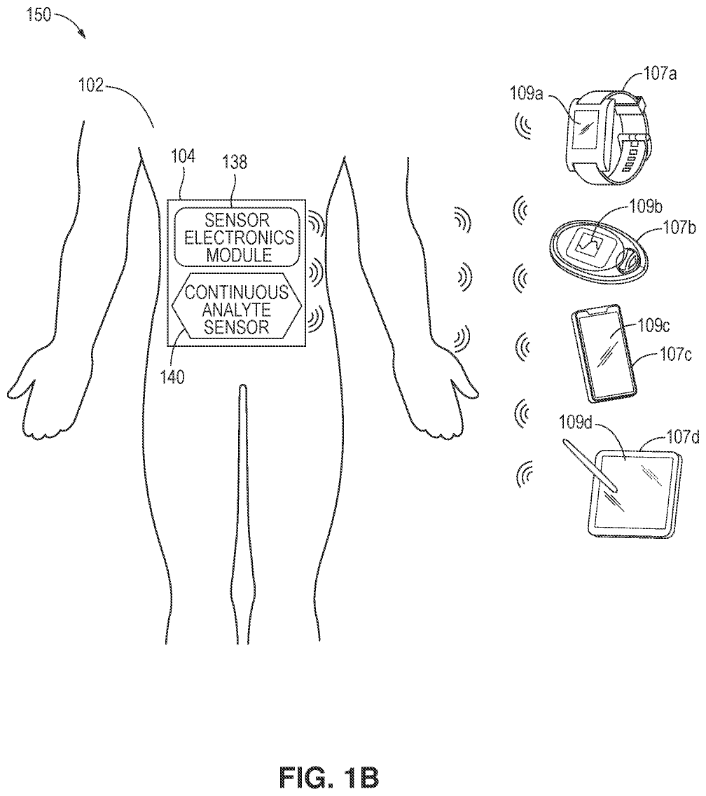

FIG. 1A illustrates an example health monitoring and support system, in accordance with certain embodiments of the disclosure. The health monitoring and support system 100 may be utilized for monitoring user health and displaying cross-feature correlation insights using various UIs to users associated with system 100. Each user of system 100, such as user 102, may interact with a mobile health application, such as mobile health application (“application”) 106 (e.g., a diabetes intervention application that provides decision support guidance), and/or a health monitoring device, such as an analyte monitoring system 104 (e.g., a glucose monitoring system). User 102, in certain embodiments, may be the patient or, in some cases, the patient's caregiver. In the embodiments described herein, the user is assumed to be the patient for simplicity only, but is not so limited. As shown, system 100 may include an analyte monitoring system 104, a mobile device 107 that executes application 106, a cross-feature correlation engine 112 (including a Data Analysis Module (DAM) 111), and a user database 110.

Analyte monitoring system 104 may be configured to generate analyte measurements (e.g., sensor data) for the user 102, e.g., on a continuous basis, and transmit the analyte measurements to the mobile device 107 for use by application 106. In some embodiments, the analyte monitoring system 104 may transmit the analyte measurements to the mobile device 107 through a wireless connection (e.g., Bluetooth connection). In certain embodiments, mobile device 107 is a smart phone. However, in certain embodiments, mobile device 107 may instead be any other type of computing device such as a laptop computer, a smart watch, a tablet, or any other computing device capable of executing application 106.

Note that, while in certain examples the analyte monitoring system 104 is assumed to be a glucose monitoring system, analyte monitoring system 104 may operate to monitor one or more additional or alternative analytes. As discussed, the term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates). Analytes can include naturally occurring substances, artificial substances, metabolites, and/or reaction products. In some embodiments, the analyte for measurement by the sensing regions, devices, and methods is albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, sodium, total protein, uric acid, metabolic markers, and drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; Cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

Application 106 may be a mobile health application that is configured to receive and analyze analyte measurements from the analyte monitoring system 104. In some embodiments, application 106 may transmit analyte measurements received from the analyte monitoring system 104 to a user database 110 (and/or the cross-feature correlation engine 112), and the user database (and/or the cross-feature correlation engine 112) may store the analyte measurements in a user profile 118 of user 102 for processing and analysis as well as for use by the cross-feature correlation engine 112 to determine cross-feature correlation insights and provide the cross-feature correlation insights to the user 102 using UIs via the application 106. In some embodiments, application 106 may store the analyte measurements in a user profile 118 of user 102 locally for processing and analysis as well as for use by the cross-feature correlation engine 112 to determine and present cross-feature correlation insights using UIs to the user 102.

In certain embodiments, cross-feature correlation engine 112 refers to a set of software instructions with one or more software modules, including a data analysis module (DAM) 111. In some embodiments, cross-feature correlation engine 112 executes entirely on one or more computing devices in a private or a public cloud. In some other embodiments, cross-feature correlation engine 112 executes partially on one or more local devices, such as mobile device 107, and partially on one or more computing devices in a private or a public cloud. In some other embodiments, cross-feature correlation engine 112 executes entirely on one or more local devices, such as mobile device 107.

As discussed in more detail herein, cross-feature correlation engine 112, may determine cross-feature correlation insights and present the cross-feature correlation insights to the user via the application 106. For example, the cross-feature correlation engine 112 may identify one or more analyte features and one or more correlative features, determine an analyte feature trend and a correlative feature trend, and determine cross-feature correlation insight(s) based on the analyte feature trend and the correlative feature trend, as further described below. In some embodiments, the cross-feature correlation engine 112 may determine cross-feature correlation insight(s) and present cross-feature correlation insights based on information including, but not limited to, information included in the user profile 118 stored in the user database 110. In some embodiments, the user profile 118 may include information collected about the user from the application 106, as further described below.

In certain embodiments, DAM 111 of decision support engine 112 may be configured to receive and/or process a set of inputs 127 (described in more detail below) (also referred to herein as “input data”) to determine one or more metrics 130 (also referred to herein as “metrics data”) that may then be used by decision support engine 112 in determining and displaying cross-feature correlation insights. Inputs 127 may be stored in the user profile 118 in the user database 110. DAM 111 can fetch inputs 127 from the user database 110 and compute a plurality of metrics 130 which can then be stored as application data 126 in the user profile 118. Such metrics 130 may include health-related metrics.

In certain embodiments, application 106 is configured to take as input information relating to user 102 and store the information in a user profile 118 for user 102 in user database 110. For example, application 106 may obtain and record user 102's demographic info 119, disease progression info 121, and/or medication info 122 in user profile 118. In certain embodiments, demographic info 119 may include one or more of the user's age, body mass index (BMI), ethnicity, gender, etc. In certain embodiments, disease progression info 121 may include information about the user 102's disease, such as, for diabetes, whether the user is Type I, Type II, pre-diabetes, or whether the user has gestational diabetes. In certain embodiments, disease progression info 121 also includes the length of time since diagnosis, the level of disease control, level of compliance with disease management therapy, predicted pancreatic function, other types of diagnosis (e.g., heart disease, obesity) or measures of health (e.g., heart rate, exercise, stress, sleep, etc.), and/or the like. In certain embodiments, medication regimen info 122 may include information about the amount and type of medication taken by user 102, such as insulin or non-insulin diabetes medications and/or non-diabetes medication taken by user 102.

In certain embodiments, application 106 may obtain demographic info 119, disease progression info 121, and/or medication info 122 from the user 102 in the form of user input or from other sources. In certain embodiments, as some of this information changes, application 106 may receive updates from the user 102 or from other sources. In certain embodiments, user profile 118 associated with the user 102, as well as other user profiles associated with other users are stored in a user database 110, which is accessible to application 106, as well as to the decision support engine 112, over one or more networks (not shown). In certain embodiments, application 106 collects inputs 127 through user 102 input and/or a plurality of other sources, including analyte monitoring system 104, other applications running on mobile device 107, and/or one or more other sensors and devices. In certain embodiments, such sensors and devices include one or more of, but are not limited to, an insulin pump, other types of analyte sensors, sensors or devices provided by mobile device 107 (e.g., accelerometer, camera, global positioning system (GPS), heart rate monitor, etc.) or other user accessories (e.g., a smart watch), or any other sensors or devices that provide relevant information about the user 102. In certain embodiments, user profile 118 also stores application configuration information indicating the current configuration of application 106, including its features and settings.

User database 110, in some embodiments, refers to a storage server that may operate in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 may include other user profiles 118 associated with a plurality of other users served by health monitoring system 100. More particularly, similar to the operations performed with respect to the user 102, the operations performed with respect to these other users may utilize an analyte monitoring system, such as analyte monitoring system 104, and also interact with the same application 106, copies of which execute on the respective mobile devices of the other users 102. For such users, user profiles 118 are similarly created and stored in user database 110.

FIG. 1B illustrates a continuous analyte monitoring system, in accordance with certain embodiments of the disclosure. The diagram 150 illustrates an example of an analyte monitoring system 104. In the example of FIG. 1B, the analyte monitoring system 104 is a glucose monitoring system. However, as described above, analyte monitoring system 104 may be configured for measuring any other analytes or a combination of multiple analytes. FIG. 1B illustrates a number of mobile devices 107a, 107b, 107c, and 107d (individually referred to as mobile device 107 and collectively referred to as mobile devices 107). Note that mobile device 107 of FIG. 1A may be any one of mobile devices 107a, 107b, 107c, or 107d. In other words, any one of mobile devices 107a, 107b, 107c, or 107d may be configured to execute application 106. The analyte monitoring system 104 may be communicatively coupled to mobile devices 107a, 107b, 107c, and/or 107d.

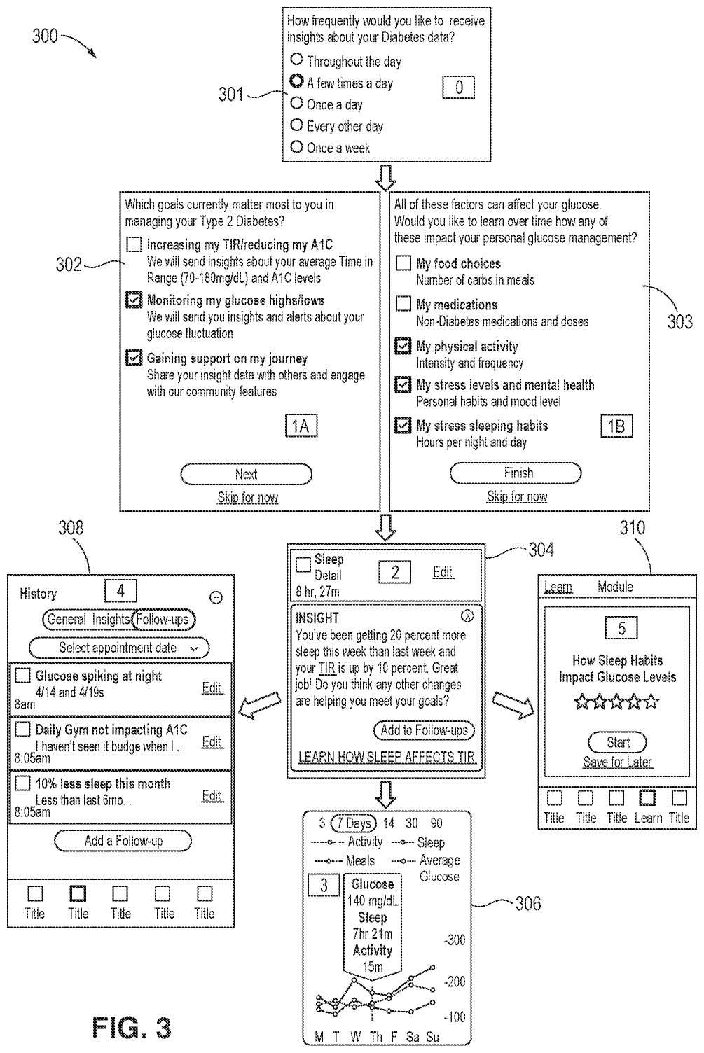

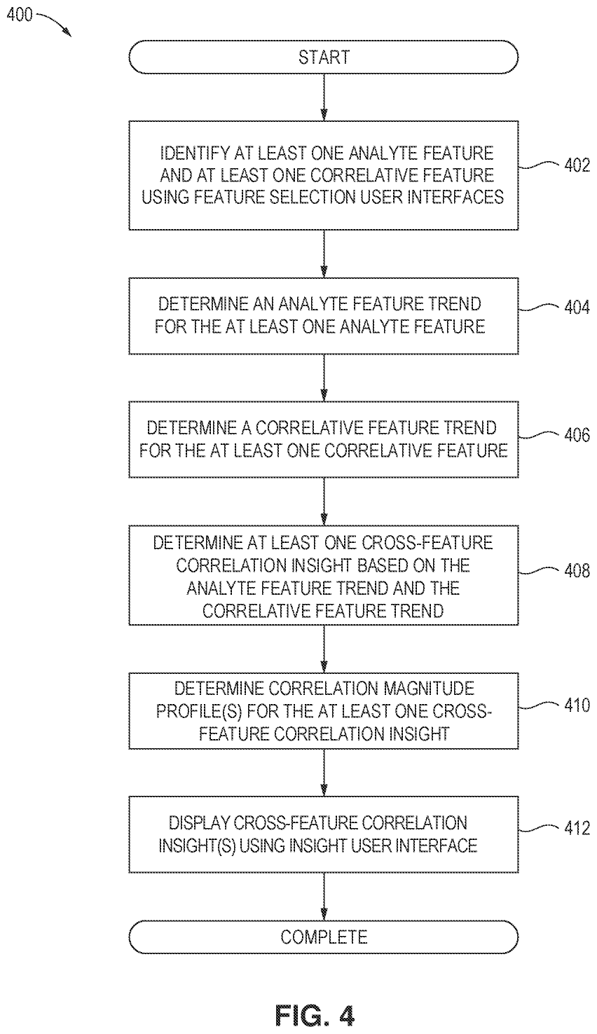

By way of an overview and an example, the analyte monitoring system 104 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices, such as mobile devices 107. Paragraphs [0137]-[0140] and FIGS. 3A, 3B, and 4 of U.S. Patent Application Publication No. 2019/0336053 further describe an on-skin sensor assembly that, in certain embodiments, may be used in connection with analyte monitoring system 104. Paragraphs [0137]-[0140] and FIGS. 3A, 3B, and 4 of U.S. Patent Application Publication No. 2019/0336053 are incorporated herein by reference.

In certain embodiments, analyte monitoring system 104 includes an analyte sensor electronics module 138 and a continuous analyte sensor 140 (e.g., a glucose sensor) associated with the analyte sensor electronics module 138. In certain embodiments, analyte sensor electronics module 138 includes electronic circuitry associated with measuring and processing analyte sensor data or information, including algorithms associated with processing and/or calibration of the analyte sensor data/information. Analyte sensor electronics module 138 may be physically/mechanically connected to the analyte sensor 140 and can be integral with (e.g., non-releasably attached to) or releasably attachable to the analyte sensor 140.

Analyte sensor electronics module 138 may also be electrically coupled to analyte sensor 140, such that the components may be electromechanically coupled to one another. Analyte sensor electronics module 138 may include hardware, firmware, and/or software that enable measurement and/or estimation of levels of the analyte in the user via analyte sensor 140 (e.g., which may be/include a glucose sensor). For example, analyte sensor electronics module 138 can include one or more potentiostats, a power source for providing power to analyte sensor 140, other components useful for signal processing and data storage, and a telemetry module for transmitting data from the sensor electronics module to various devices including, but not limited to, one or more display devices (e.g., the user's mobile device 107), user database 110, decision support engine 112, etc. Electronics can be affixed to a printed circuit board (PCB) within analyte monitoring system 104, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 138 may include sensor electronics that are configured to process sensor information, such as sensor data, and generate transformed sensor data and displayable sensor information. Examples of systems and methods for processing analyte data are described in more detail herein and in U.S. Pat. Nos. 7,310,544 and 6,931,327 and U.S. Patent Application Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

Analyte sensor 140 is configured to measure a concentration or level of the analyte in the user 102. The term analyte is further defined by paragraph [0117] of U.S. App. No. 2019/0336053. Paragraph [0117] of U.S. App. No. 2019/0336053 is incorporated herein by reference. In some embodiments, analyte sensor 140 comprises a continuous analyte sensor, such as a subcutaneous, transdermal (e.g., transcutaneous), or intravascular device. In some embodiments, analyte sensor 140 can analyze a plurality of intermittent blood samples. Analyte sensor 140 can use any method of analyte-measurement, including enzymatic, chemical, physical, electrochemical, spectrophotometric, polarimetric, calorimetric, iontophoretic, radiometric, immunochemical, and the like. Additional details relating to a continuous analyte sensor, such as a continuous glucose sensor, are provided in paragraphs [0072]-[0076] of U.S. Pat. No. 9,445,445. Paragraphs [0072]-[0076] of U.S. Pat. No. 9,445,445 are incorporated herein by reference.

With further reference to FIG. 1B, mobile devices 107 can be configured for displaying (and/or alarming) displayable sensor information that may be transmitted by sensor electronics module 138 (e.g., in a customized data package that is transmitted to the display devices based on their respective preferences). Each of mobile devices 107a, 107b, 107c, and/or 107d may respectively include a display such as touchscreen display 109a, 109b, 109c, and/or 109d for displaying a graphical user interface (e.g., of application 106) for presenting sensor information and/or analyte data to user 102 and/or receiving inputs from user 102. In certain embodiments, the mobile devices 107 may include other types of user interfaces such as voice user interface instead of or in addition to a touchscreen display for communicating sensor information to user 102 of the mobile device 107 and/or receiving user inputs. In certain embodiments, one, some, or all of mobile devices 107 may be configured to display or otherwise communicate the sensor information as it is communicated from sensor electronics module 138 (e.g., in a data package that is transmitted to respective display devices), without any additional prospective processing required for calibration and/or real-time display of the sensor data.

The mobile devices 107 may include a custom or proprietary display device, for example, analyte display device 107b, especially designed for displaying certain types of displayable sensor information associated with analyte data received from sensor electronics module 138 (e.g., a numerical value and/or an arrow, in certain embodiments). In certain embodiments, one of the mobile devices 107 includes a mobile phone, such as a smartphone that uses an Android, iOS, or another operating system configured to display a graphical representation of the continuous sensor data (e.g., including current and/or historic data). As further described herein, mobile devices 107 may be configured for dynamic determination and presentation of cross-feature correlation insights for continuous analyte data.

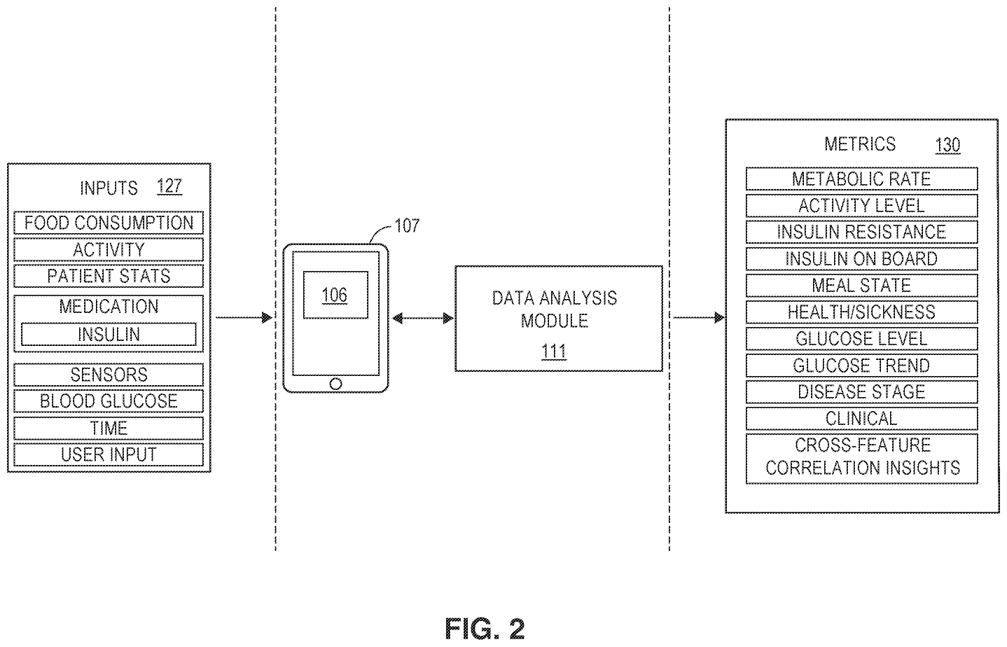

Example inputs and example metrics that are generated based on the inputs in accordance with certain embodiments of the disclosure are illustrated in FIG. 2. FIG. 2 illustrates example inputs 127 on the left, application 106 and DAM 111 in the middle, and example metrics 130 on the right. In certain embodiments, application 106 may obtain inputs 127 through one or more channels (e.g., manual user input, sensors, various applications executing on mobile device 107, etc.). Inputs 127 may be further processed by DAM 111 to output a plurality of metrics, such as metrics 130. Further, inputs (e.g., inputs 127) and metrics (e.g., metrics 130) may be used by the DAM 111 and/or any computing device in the system 100 to perform various processes in determining and displaying cross-feature correlation insights to users, as further described below. Any of inputs 127 may be used for computing any of metrics 130. In certain embodiments, each one of metrics 130 may correspond to one or more values, e.g., discrete numerical values, ranges, or qualitative values (high/medium/low or stable/unstable).

In certain embodiments, inputs 127 include food consumption information. Food consumption information may include information about one or more of meals, snacks, and/or beverages, such as one or more of the size, content (carbohydrate, fat, protein, etc.), sequence of consumption, and time of consumption. In certain embodiments, food consumption may be provided by the user through manual entry, by providing a photograph through an application that is configured to recognize food types and quantities, and/or by scanning a bar code or menu. In various examples, meal size may be manually entered as one or more of calories, quantity (e.g., ‘three cookies’), menu items (e.g., ‘Royale with Cheese’), and/or food exchanges (1 fruit, 1 dairy). In some examples, meals may also be entered with the user's typical items or combinations for this time or context (e.g., workday breakfast at home, weekend brunch at restaurant). In some examples, meal information may be received via a convenient user interface provided by application 106.

In certain embodiments, inputs 127 include activity information. Activity information may be provided, for example, by an accelerometer sensor on a wearable device such as a watch, fitness tracker, and/or patch. In certain embodiments, activity information may also be provided through manual input by user 102. Activity information may include exercise related information, sleep information, and other types of information related to the user's activity or lack thereof.

In certain embodiments, inputs 127 include patient statistics, such as one or more of age, height, weight, body mass index, body composition (e.g., % body fat), stature, build, or other information. Patient statistics may be provided through a user interface, by interfacing with an electronic source such as an electronic medical record, and/or from measurement devices. The measurement devices may include one or more of a wireless, e.g., Bluetooth-enabled, weight scale and/or camera, which may, for example, communicate with the mobile device 107 to provide patient data.

In certain embodiments, inputs 127 include information relating to the user's medication intake. For example, the user's medication intake may include the user's insulin delivery. Such information may be received, via a wireless connection on a smart pen, via user input, and/or from an insulin pump. Insulin delivery information may include one or more of insulin volume, time of delivery, etc. Other configurations, such as insulin action time or duration of insulin action, may also be received as inputs.

In certain embodiments, inputs 127 include information received from sensors, such as physiologic sensors, which may detect one or more of heart rate, respiration, oxygen saturation, body temperature, etc. (e.g., to detect illness, stress levels, etc.).

In certain embodiments, inputs 127 include glucose information. Such information may be provided as input, for example through analyte monitoring system 104. In certain embodiments, blood glucose information may be received from one or more of smart pill dispensers that track when the user takes medicine, a blood ketone meter, a laboratory-measured, or estimated A1C, other measures of long-term control, or sensors that measure peripheral neuropathy using tactile response, such as by using haptic features of a smartphone, or a specialty device.

In certain embodiments, inputs 127 include time, such as time of day, or time from a real-time clock.

As described above, in certain embodiments, DAM 111 determines or computes metrics 130 based on inputs 127 associated with user 102. An example list of metrics 130 is illustrated in FIG. 2. In certain embodiments, metrics 130 determined or computed by DAM 111 include metabolic rate. Metabolic rate is a metric that may indicate or include a basal metabolic rate (e.g., energy consumed at rest) and/or an active metabolism, e.g., energy consumed by activity, such as exercise or exertion. In some examples, basal metabolic rate and active metabolism may be tracked as separate metric. In certain embodiments, the metabolic rate may be calculated by DAM 111 based on one or more of inputs 127, such as one or more of activity information, sensor input, time, user input, etc.

In certain embodiments, metrics 130 determined or computed by DAM 111 include an activity level metric. The activity level metric may indicate a level of activity of the user. In certain embodiments, the activity level metric be determined, for example based on input from an activity sensor or other physiologic sensors. In certain embodiments, the activity level metric may be calculated by DAM 111 based on one or more of inputs 210, such as one or more of activity information, sensor input, time, user input, etc. Activity level may indicate whether the user is exercising, at rest, sleeping, etc.

In certain embodiments, metrics 130 determined or computed by DAM 111 include an insulin sensitivity metric (also referred to herein as an “insulin resistance”). The insulin sensitivity metric may be determined using historical data, real-time data, or a combination thereof, and may, for example, be based upon one or more inputs 127, such as one or more of food consumption information, blood glucose information, insulin delivery information, the resulting glucose levels, etc. In certain embodiments, the insulin on board metric may be determined using insulin delivery information, and/or known or learned (e.g., from patient data) insulin time action profiles, which may account for both basal metabolic rate (e.g., update of insulin to maintain operation of the body) and insulin usage driven by activity or food consumption.

In certain embodiments, metrics 130 determined or computed by DAM 111 include a meal state metric. The meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first.).

In certain embodiments, metrics 130 determined or computed by DAM 111 include health and sickness metrics. Health and sickness metrics may be determined, for example, based on one or more of user input (e.g., pregnancy information or known sickness information), from physiologic sensors (e.g., temperature), activity sensors, or a combination thereof. In certain embodiments, based on the values of the health and sickness metrics, for example, the user's state may be defined as being one or more of healthy, ill, rested, or exhausted. In certain embodiments, health and sickness metric may indicate the user's heart rate, stress level, etc.

In certain embodiments, metrics 130 determined or computed by DAM 111 include glucose level metrics. Glucose level metrics may be determined from sensor information (e.g., blood glucose information obtained from analyte monitoring system 104). In some examples, a glucose level metric may also be determined, for example, based upon historical information about glucose levels in particular situations, e.g., given a combination of food consumption, insulin, and/or activity. In certain embodiments, a blood glucose trend may be determined based on the glucose level over a certain period of time.

In certain embodiments, metrics 130 determined or computed by DAM 111 include a disease stage. For example disease stages for Type II diabetics may include a pre-diabetic stage, an oral treatment stage, and a basal insulin treatment stage. In certain embodiments, degree of glycemic control (not shown) may also be determined as an outcome metric, and may be based, for example, on one or more of glucose levels, variation in glucose level, or insulin dosing patterns.

In certain embodiments, metrics 130 determined or computed by DAM 111 include clinical metrics. Clinical metrics generally indicate a clinical state a user is in with respect to one or more conditions of the user, such as diabetes. For example, in the case of diabetes, clinical metrics may be determined based on glycemic measurements, including one or more of A1C, trends in A1C, time in range, time spent below a threshold level, time spent above a threshold level, and/or other metrics derived from blood glucose values. In certain embodiments, clinical metrics may also include one or more of estimated A1C, glycemic variability, hypoglycemia, and/or health indicator (time magnitude out of target zone).

In certain embodiments, metrics 130 determined or computed by DAM 111 include cross-feature correlation insights. A cross-feature correlation insight provides the user with information based on a correlation determined between an analyte feature and a correlative feature. As further described below, certain embodiments described herein relate to processes that include identifying one or more analyte features and one or more correlative features, identifying an analyte feature trend and a correlative feature trend, as well as identifying cross-feature correlation insights. In certain embodiments, analyte and/or correlative features may include or be based on any input data (e.g., inputs 127) and/or metrics (e.g., metrics 130). As further described below, a cross-feature correlation insight is a determined correlation between an analyte feature of a user and a correlative feature of the user. Examples of analyte features include a time-in-range, an A1C analyte feature, a glucose high feature, a glucose low feature, etc. In certain embodiments, analyte features may be based on various data including, but not limited to, inputs 127. An analyte feature trend is any trend related to an analyte feature, as further described below. In certain embodiments, analyte feature trends may be based on various data including, but not limited to, metrics 130.

Further, a correlative feature is a feature whose potential correlation with an analyte feature may be analyzed. Examples of correlative features include a sleep level, a medication intake, a food intake, and an exercise level, stress, heartrate, etc. A correlative feature trend is any trend related to a correlative feature, as further described below. In certain embodiments, correlative feature trends may be based on various data including, but not limited to, metrics 130. In addition, cross-feature correlation insights may be determined between one or more analyte features of the user and one or more correlative features of the user, as further described below. In certain embodiments, cross-feature correlative insights may be stored as metrics 130. In various embodiments, the dynamic determination and presentation of cross-feature correlation insights may utilize various UIs, as further described below.

Exemplary User Interfaces for Use with Dynamic Determination and Presentation of Cross-Feature Correlation Insights

FIG. 3 is a flow diagram illustrating exemplary user interfaces (UIs) for use with dynamic determination and presentation of cross-feature correlation insights, in accordance with certain embodiments of the disclosure. The flow diagram 300 includes five types of UIs that may be utilized in the dynamic determination and presentation of cross-feature correlation insights. In certain embodiments, the five types of UIs may include (1) a set of feature selection UIs, (2) an insight flagging UI, (3) a cross-temporal insight interaction UI, (4) a set of flagged insight engagement UIs, and (5) a set of content engagement UIs. The various UIs may be generated and presented using a software application (e.g., application 106) that executes on a computing device (e.g., mobile device 107). In certain embodiments, the flow diagram 300 may include an insight reporting configuration UI 301 for configuring the insight reporting settings such as the frequency of reporting insights to the user and other aspects of the reporting settings, etc. However, in some embodiments, the insight reporting configuration UI 301 may be omitted. In such embodiments, the flow diagram 300 may begin with the set of feature selection UIs, as further described below.

Using the insight reporting configuration UI 301, the computing device may present insight reporting settings for the user's configuration. For example, the insight reporting configuration UI 301 includes a prompt text element presenting a general question related to how frequently the user would like to receive cross-feature correlation insights. The insight reporting configuration UI 301 may also include one or more checkbox elements that enable the user to select an answer to the question presented. In the depicted example, the user is presented with a first checkbox and a first text element (e.g., “Throughout the day”), a second checkbox and a second text element (e.g., “A few times a day”), a third checkbox and a third text element (e.g., “Once a day”), a fourth checkbox and a fourth text element (e.g., “Every other day”), and a firth checkbox and a firth text element (e.g., “Once a week”). The computing system may then configure the frequency of reporting cross-feature correlation insights to the user based on the user selection.

Next, the computing device may present a set of feature selection UIs (e.g., analyte feature selection UI 302 and the correlative feature selection UI 303) that enable user selection of analyte features and correlative features. For example, the analyte feature selection UI 302 enables user selection of one or more analyte features with respect to which cross-feature correlation insights are determined. The analyte feature selection UI 302 includes a prompt text element presenting a question related to analyte features. Further, the analyte feature selection UI 302 also includes one or more checkbox elements that enable selecting analyte features as well as other user goals (e.g., in the depicted example, the user goal of gaining support on their journey). In the depicted example, a user is presented with (1) a first checkbox element and first text element that is associated with a combination of a time-in-range analyte feature and an A1C analyte feature, (2) a second checkbox element and a second text element that is associated with a combination of a glucose high analyte feature and a glucose low analyte feature, and (3) a third checkbox element and a third text element that is associated with the user goal of gaining support on their journey. Further, the analyte feature selection UI 302 may also include a button element (e.g., “Next” button element) that enables transitioning from the analyte feature selection UI 302 to a subsequent UI (e.g., to a correlative feature selection UI 303).

Using the correlative feature selection UI 303, the computing device may enable user selection of one or more correlative features with respect to which cross-feature correlation insights are determined. For example, the correlative feature selection UI 303 includes a prompt text element presenting a question related to correlative features. Further, the correlative feature selection UI 303 also includes checkbox elements that enable selecting correlative features. In the depicted example, the correlative feature selection UI 303 incudes (1) a first checkbox element and first text element associated with a food-intake-related correlative feature, (2) a second checkbox element and second text element associated with a medication-intake-related correlative feature, (3) a third checkbox element and third text element associated with a physical-activity-related correlative feature, (4) a fourth checkbox element and fourth text element associated with a stress-level-related correlative feature, and (5) a fifth checkbox element and first text element associated with a sleeping-habit-related correlative feature. In addition, the correlative feature selection UI 303 also includes a button element (e.g., the “Finish” button element) that enables transitioning from the correlative feature selection UI 303 to a subsequent UI such as the insight flagging UI 304.

In certain embodiments, displaying the set of feature selection UIs may take place during a sensor warm-up session, which refers to a period of time subsequent to the software application 106 and the continuous analyte monitoring system 104 connecting for the first time. During this period, the continuous analyte monitoring system 104 may be referred to as acclimatizing. In such embodiments, although analyte data may be measured by the continuous analyte monitoring system 104 and transmitted to the software application 106 during this period, the measurements may not be displayed or reported to the user.

Using the insight flagging UI 304, after determining cross-feature correlation insights between the analyte features and the correlative features, the computing device may display the determined cross-feature correlation insight to the user enabling the user to flag a cross-feature correlation insight, as well as to initiate engagement with educational content associated with the cross-feature correlation insight, as further described below. For example, the insight flagging UI 304 includes a textbox element that depicts notification for content data other than the cross-feature correlation insight (e.g., “Sleep”). Further, the insight flagging UI 304 includes an insight interaction element that enables user engagement with the cross-feature correlation insight. As depicted, the insight interaction element depicts an insight textbox element that provides information including the correlation magnitude profile for the cross-feature correlation insight, an upper button element (e.g., the “Add to Follow-ups” button element) that enables flagging the depicted cross-feature correlation insight for discussion in a follow-up medical appointment, and a lower button element (e.g., the “LEARN HOW SLEEP AFFECTS TIR” button element) that enables the user to engage with educational content related to the depicted cross-feature correlation insight.

Using the cross-temporal insight interaction UI 306, the computing device may enable the user to view correlation magnitude data for a flagged cross-feature correlation insight with respect to various user-selected correlation periods, as further described below. For example, the cross-temporal insight interaction UI 306 includes a trend line depiction element that depicts a set of fluctuation trend lines for a set of selected features. Further, the cross-temporal insight interaction UI 306 includes a first layer of button elements that enable selecting a correlation period from a set of available correlation periods. In certain embodiments, a default value of the selected correlation period for a depicted cross-feature correlation insight may be determined based on correlation scores associated with the depicted cross-feature correlation insight across a set of correlation periods. The cross-temporal insight also includes a second layer of button elements that enable selecting features whose fluctuation trend lines with respect to the selected correlation period are depicted in the trend line depiction element.

Using the flagged insight engagement UI 308, the computing device may enable the user to edit cross-feature correlation insights that were previously flagged using the insight flagging UI and to assign various metadata fields to the flagged cross-feature correlation insights, as further described below. For example, the flagged insight engagement UI 308 includes a navigation element (e.g., the selected “Follow-ups” navigation element) that allows the user to navigate between various UIs as described herein. The flagged insight engagement UI 308 also includes an upper button element (e.g., the “Select appointment date” button element) that enables assigning a future timestamp (e.g., an appointment date) for the flagged cross-feature correlation insights that are being displayed by the flagged insight engagement UI 308.

The flagged insight engagement UI 308 also includes a modification button element (e.g., the “Edit” button element) for each depicted cross-feature correlation insight that enables changing the description of the cross-feature correlation insight and adding explanatory metadata to the cross-feature correlation insight. For example, the flagged insight engagement UI 308 may include (1) a first cross-feature correlation insight (e.g., “Glucose spiking at night”) and a first modification button element, (2) a second cross-feature correlation insight (e.g., “Daily Gym not impacting A1C”) and a second modification button element, and (3) a third cross-feature correlation insight (e.g., “10% less sleep this month”) and a third modification button element. Further, the flagged insight engagement UI 308 also includes a lower button element (e.g., the “Add to Follow-up” button element) that enables flagging the depicted cross-feature correlation insight for discussion in a follow-up medical appointment.

Using the content engagement UI 310, the computing device may enable the user to engage with educational content selected for the user, including educational content selected for the user based on the flagged cross-feature correlation insights, as further described below. For example, the content engagement UI 310 includes an educational content text element that is a title of educational content being presented (e.g., “How Sleep Habits Impact Glucose Levels”). The user may engage with the educational content using a start element and the user may rate the particular educational content using a rating element (e.g., stars from 1 to 5).

### Example Processes for Dynamic Determination and Presentation of Cross-Feature Correlation Insights Using User Interfaces

FIG. 4 is a flow diagram illustrating an exemplary process 400 for dynamic determination and presentation of cross-feature correlation, in accordance with certain embodiments of the disclosure.

At block 402, the process 400 includes identifying at least one analyte feature and at least one correlative feature. As described further above, analyte features are features that are determined based on sensor data generated by the analyte sensor 140 (e.g., time-in-range feature, A1C analyte feature, a glucose high feature, a glucose low feature, etc.). Correlative features are features that may have a potential correlation to an analyte feature (e.g., a sleep level feature, a medication intake feature, a food intake feature, an exercise level feature, activity feature, stress level feature, heart rate feature, etc.).

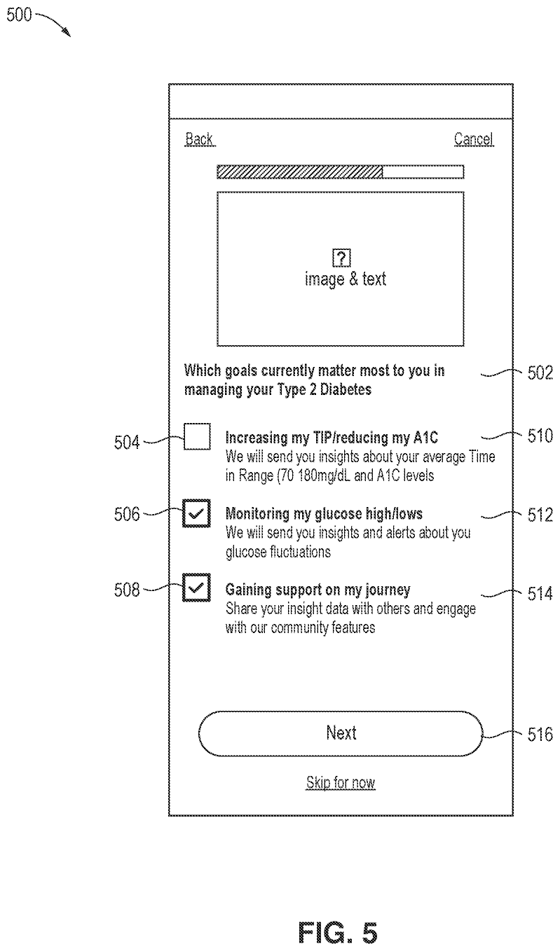

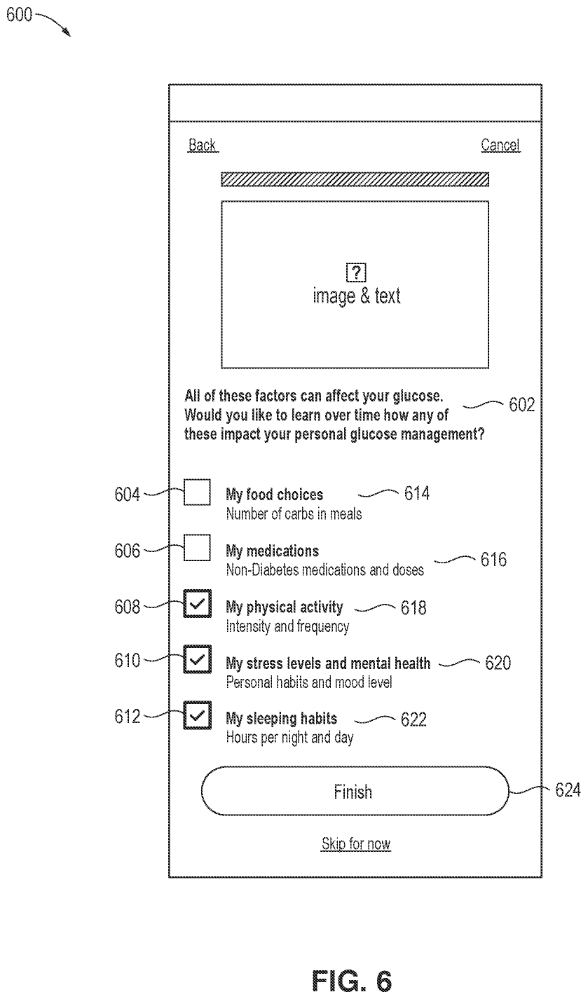

In certain embodiments, the at least one analyte feature and the at least one correlative feature may be identified by providing the user with feature selection UIs. For example, a computing device (e.g., the mobile device 107 that executes the software application 106) may be configured to generate and display one or more feature selection UIs, such as the feature selection UIs (e.g., the analyte feature selection UI 302, and/or the correlative feature selection UI 303) of FIG. 3. The feature selection UIs enable a user to select analyte features and correlative features that are then used to determine cross-feature correlation insights, as further described below. In some embodiments, two different feature selection UIs are presented to the user: an analyte feature selection UI and a correlative feature selection UI, as further described below in reference to FIGS. 5 and 6, respectively.

At block 404, the process 400 includes determining an analyte feature trend for each of the identified at least one analyte features. In some embodiments, the application 106 and/or the DAM 11 may determine an analyte feature trend by determining changes in values of an analyte feature over time. In various embodiments, the analyte feature trend describes fluctuations of the analyte feature over a period of time. For example, when the identified analyte feature (from block 402) is the time-in-range feature, the analyte feature trend includes trends in time-in-range fluctuations over a period of time. In another example, when the identified analyte feature (from block 402) is the A1C feature, the analyte feature trend includes trends in A1C fluctuations over a period of time.

In yet another example, when the identified analyte feature (from block 402) is the glucose high feature, the analyte feature trend includes trends in glucose high fluctuations over a period of time, where glucose high fluctuations refer to changes in the maximum glucose readings of a user across a sequence of time periods. In a further example, when the identified analyte feature (from block 402) is the low glucose feature, the analyte feature trend includes trends in glucose low fluctuations over a period of time, where glucose low fluctuations refer to changes in minimum glucose readings of a user across a sequence of time periods. In some embodiments, determining analyte feature trends includes receiving, from the analyte monitoring system 104, sensor data representative of the analyte levels of a user so that the sensor data may be utilized to determine fluctuations of the analyte over a period of time.

At block 406, the process 400 includes determining a correlative feature trend for each of the identified at least one correlative features. In some embodiments, the application 106 and/or the DAM 11 may determine a correlative feature trend by determining changes in values of a correlative feature over time. In various embodiments, the correlative feature trend describes fluctuations of the correlative feature over a period of time. For example, when the identified correlative feature (from block 402) is the sleep level feature, the correlative feature trends may include trends in sleep length fluctuations over a period of time, sleep quality fluctuations over a period of time, etc. In another example, when the identified correlative feature (from block 402) is the medication intake feature (e.g., insulin intake feature), the correlative feature trend includes trends in medication (e.g., insulin) intake amounts over a period of time. In a further example, when the identified correlative feature (from block 402) is the food intake feature, the correlative feature trend includes trends in food intake amounts over a period of time.

At block 408, the process 400 includes determining at least one cross-feature correlation insight based on the identified analyte feature trend and the identified correlative feature trend. In certain embodiments, the computing device may determine one or more cross-feature correlation insights by determining one or more correlations between the determined analyte feature trend and the determined correlative feature trend, as further described above. In various embodiments, a cross-feature correlation insight is representative of a correlation between a respective analyte feature from a set of analyte features and a respective correlative feature from a set of correlative features.

In some embodiments, determining that a correlation exists between a respective analyte feature and a respective correlative feature includes determining that the analyte feature trend for the respective analyte feature and the correlative feature trend for the respective correlative trend are correlated across at least T correlation periods (where T may be a threshold value that may be predetermined, set by the user, set by the application, etc.). For example, the computing device (e.g., the mobile device or a back end server) determines correlation scores for an analyte feature trend and a correlative feature trend across different correlation periods until either the determined correlation scores comprise T correlation scores that satisfy a correlation score threshold or correlation scores are determined across all available correlation periods without identifying T threshold-satisfying correlation scores. In such examples, if T threshold-satisfying correlation scores are determined, then the computing device determines that a correlation exists between the respective analyte feature and the respective correlative feature. However, if correlation scores are determined across all available correlation periods without identifying T threshold-satisfying correlation scores, then the computing device determines that a correlation does not exist between the respective analyte feature and the respective correlative feature.

In other embodiments, determining at least one cross-feature correlation insight may include the computing device determining whether a correlation exists between a respective analyte feature and a respective correlative feature by determining a set of correlation scores for the respective analyte feature trend and the respective correlative feature trend across a set of correlation periods. In such embodiments, the computing device determines that the respective analyte feature and the respective correlative feature are correlated when a central tendency measure (e.g., a mean measure, a median measure, etc.) for the correlation scores satisfies (e.g., exceeds) a first threshold value, and a statistical deviation measure (e.g., a variance measure, a standard deviation measure, etc.) for the correlation scores fails to satisfy (e.g., fails to exceed) a second threshold value.

In further embodiments, determining at least one cross-feature correlation insight may include the computing device determining whether a correlation exists between a respective analyte feature and a respective correlative feature by determining a set of correlation scores for the respective analyte feature trend and the respective correlative feature trend across a set of correlation periods and providing the correlation scores as input data to a machine learning model. In such embodiments, the machine learning model may be configured to determine an output score for the respective analyte feature trend and the respective correlative feature. For example, the output score may describe the predicted/computed likelihood that the respective analyte feature and the respective correlative feature are statistically correlated. In another example, the output score may describe the predicted/computed likelihood that the respective analyte feature and the respective correlative feature are statistically correlated and the statistical correlation is likely to be of interest to the user. In such examples, the computing device may determine that the respective analyte feature and the respective correlative feature are correlated if the output score generated by the machine learning model satisfies a threshold value.

In still further embodiments, determining at least one cross-feature correlation insight may include the computing device determining whether a correlation exists between a respective analyte feature and a respective correlative feature by determining a set of analyte feature trend segments of the respective analyte feature trend across the set of available correlation periods. In such embodiments, the computing device may also determine a set of correlative feature trend segments of the respective correlative feature trend across the set of available correlation periods. Further, the computing device may compare the analyte feature trend segment and the correlative feature trend segment for each correlation period using a time-series comparison model to determine a correlation score for the correlation period. In such embodiments, the computing device may determine that the respective analyte feature and the respective correlative feature are correlated if the determined correlation scores include at least T correlation scores.

In still further embodiments, determining at least one cross-feature correlation insight may include the computing device determining whether a correlation exists between a respective analyte feature and a respective correlative feature by determining a set of analyte feature trend segments of the respective analyte feature trend across the set of available correlation periods. Then, the computing device may determine a set of correlative feature trend segments of the respective correlative feature trend across the set of available correlation periods. In such embodiments, the computing device may compare the analyte feature trend segment and the correlative feature trend segment for each correlation period using a time-series comparison model to determine a correlation score for the correlation period. In such embodiments, the computing device may determine that the respective analyte feature and the respective correlative feature are correlated when a central tendency measure (e.g., a mean measure, a median measure, etc.) for correlation scores associated satisfies (e.g., exceeds) a first threshold value and a statistical deviation measure (e.g., a variance measure, a standard deviation measure, etc.) for correlation scores fails to satisfy (e.g., fails to exceed) a second threshold value.

At block 410, the process 400 includes determining correlation magnitude profiles for each cross-feature correlation insight across available time periods. In this step, the computing device determines how the correlation magnitude of each insight changes as the user modifies a correlation period of interest (e.g., from a weekly correlation period to a monthly correlation period). In various embodiments, each correlation magnitude profile is associated with a respective cross-feature correlation insight and a respective correlation period and describes correlation magnitude data for the respective cross-feature correlation insight over the respective correlation period. For example, each correlation magnitude profile describes: (i) a magnitude of change in a respective analyte feature for the respective cross-feature correlation insight between a current time period whose length corresponds to the respective correlation period and a historical (e.g., an immediately preceding) time period whose length corresponds to the respective correlation period, and (ii) a magnitude of change in a respective correlative feature between a current time period whose length corresponds to the respective correlation period and a historical (e.g., an immediately preceding) time period whose length corresponds to the respective correlation period.

In some embodiments, a correlation magnitude profile may describe a correlation score for an analyte feature trend segment and a correlative feature trend segment, where the analyte feature trend segment is a segment of the analyte feature trend associated with a respective cross-feature correlation insight in relation to a respective correlation period, and the correlative feature trend segment is a segment of the correlative feature trend associated with a respective cross-feature correlation insight in relation to a respective correlation period.

In some embodiments, a correlation magnitude profile may describe a correlation score for an analyte feature trend segment and a correlative feature trend segment, where the analyte feature trend segment is a segment of the analyte feature trend associated with a respective cross-feature correlation insight in relation to a respective correlation period, and the correlative feature trend segment is a segment of the correlative feature trend associated with a respective cross-feature correlation insight in relation to a respective correlation period. In such embodiments, the correlation scores for the respective cross-feature correlation insights are normalized.

At block 412, the process 400 may include displaying cross-feature correlation insights using insight UIs. For example, one or more cross-feature correlation insights may be provided using the insight flagging UI and the correlation magnitude profiles may be provided using a cross-temporal insight interaction UI. Further, user engagement with the cross-feature correlation insights may be provided using flagged insight engagement UIs. In addition, educational content associated with the cross-feature correlation insight may be provided using the content engagement UIs, as further described below.

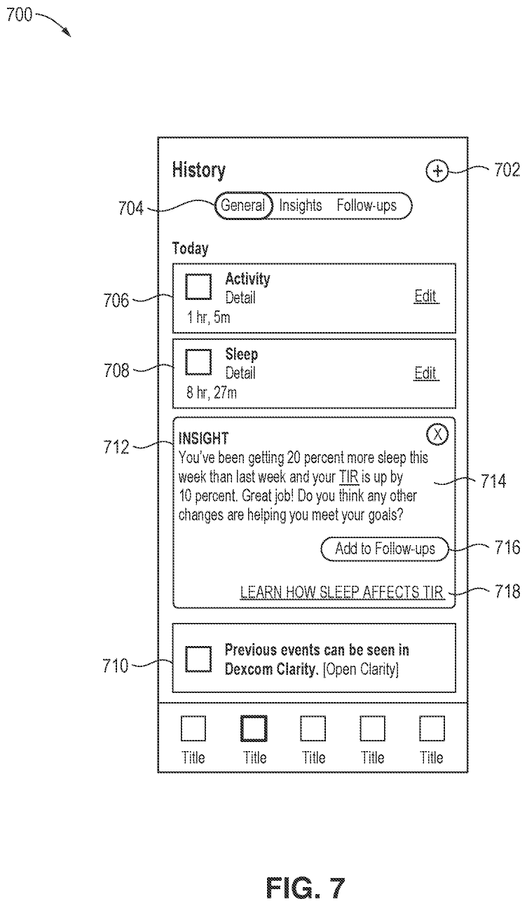

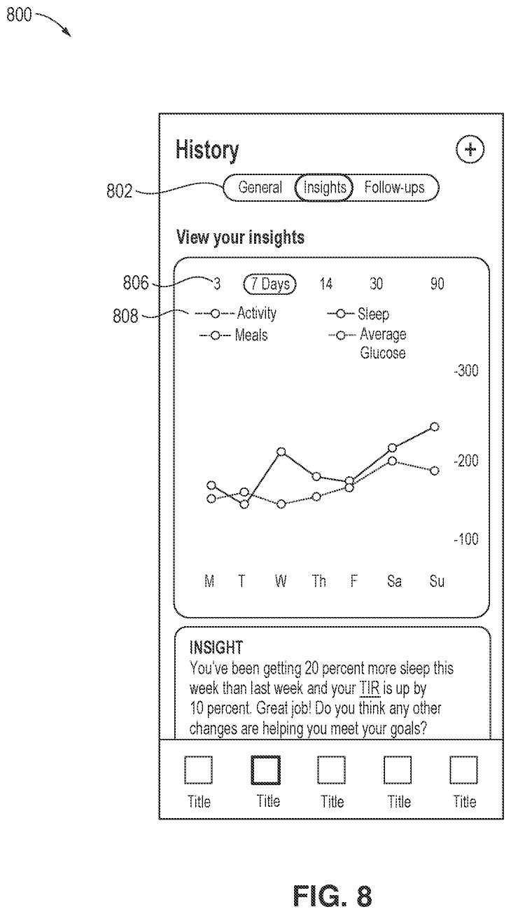

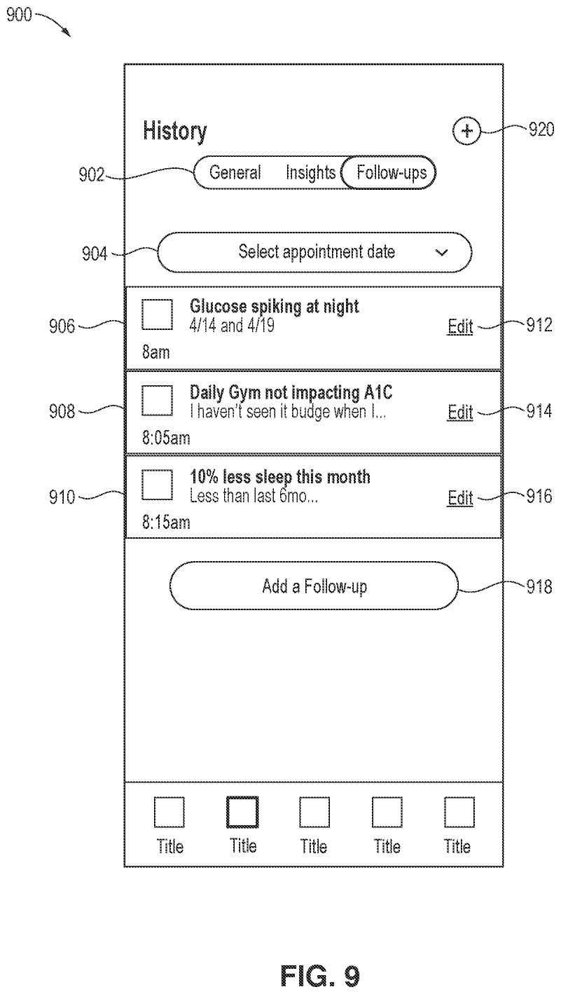

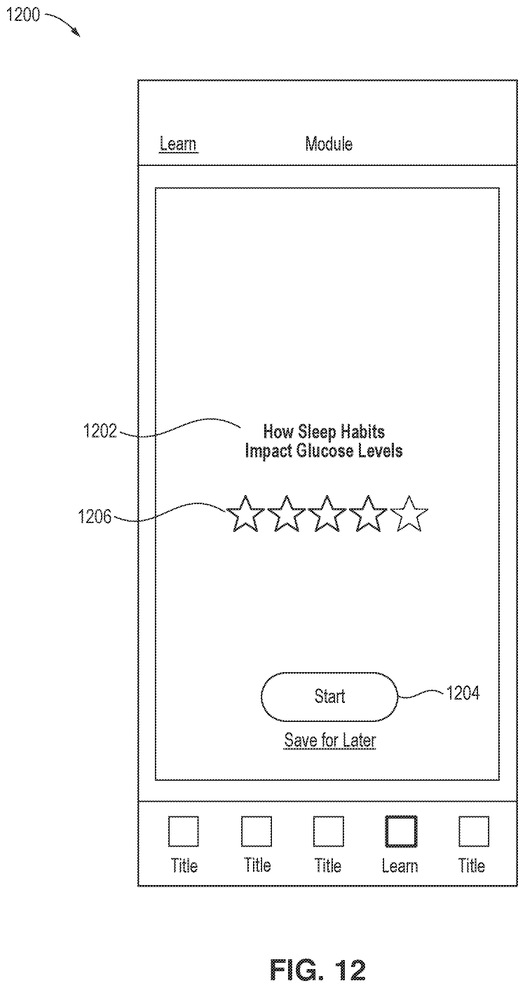

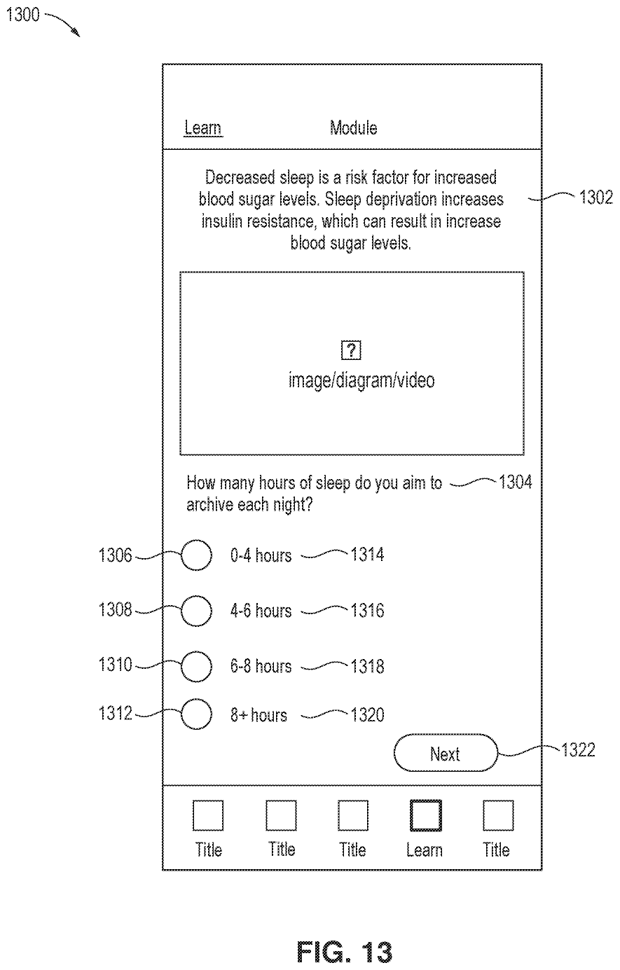

Below, block 402 is described in more detail and by reference to subsequent FIGS. 5-6 illustrating feature selection UIs. In particular, block 402 is described in more detail by reference to the analyte feature selection UI (as illustrated in FIG. 5) and the correlative feature selection UI (as illustrated in FIG. 6). In addition, block 412 is described in more detail and by reference to subsequent FIGS. 7-13 illustrating insight UIs. In particular, block 412 is described in more detail by reference to the insight flagging UI (as illustrated in FIG. 7), the cross-temporal insight interaction UI (as illustrated in FIG. 8), the flagged insight engagement UIs (as illustrated in FIGS. 9-11), and the content engagement UIs (FIGS. 12-13).

### 1. Block 402: Identifying Analyte and Correlative Features Using Feature Selection UIs

As described above in reference to FIG. 4, at block 402, the process 400 may include identifying at least one analyte feature and at least one correlative feature. In some embodiments, the at least one analyte feature may be identified using an analyte feature selection UI. In some embodiments, the at least one correlative feature may be identified using a correlate feature selection UI.

a. Analyte Feature Selection UI

FIG. 5 illustrates an analyte feature selection UI for identifying (block 402) at least one analyte feature, in accordance with certain embodiments of the disclosure. The analyte feature selection UI 500 enables user selection of one or more analyte features with respect to which cross-feature correlation insights are determined. Analyte feature values are determined based on sensor data provided by analyte monitoring system 104. In reference to FIG. 5, the analyte feature selection UI 500 includes a prompt text element 502 presenting a question related to analyte features. Further, the analyte feature selection UI 500 may also include one or more checkbox elements that enable selecting analyte features as well as other user goals (e.g., in the depicted example, the user goal of gaining support on their journey). In the depicted example, a user is presented with a first checkbox element 504 and first text element 510 that is associated with a combination of a time-in-range analyte feature and an A1C analyte feature, a second checkbox element 506 and a second text element 512 that is associated with a combination of a glucose high analyte feature and a glucose low analyte feature, and a third checkbox element 508 and a third text element 514 that is associated with the user goal of gaining support on their journey. Further, the analyte feature selection UI 500 may also include a button element 516 (e.g., “Next” button element) that enables transitioning from the analyte feature selection UI 500 to a subsequent UI (e.g., to a correlative feature selection UI).

b. Correlative Feature Selection UI

FIG. 6 illustrates a correlative feature selection UI for identifying (block 402) at least one correlative feature, in accordance with certain embodiments of the disclosure. The correlative feature selection UI 600 enables user selection of one or more correlative features with respect to which cross-feature correlation insights are determined. Correlative features may be features whose correlations with analyte features are determined and presented using cross-feature correlation insights. In some embodiments, correlative features could be based on manually or automatically inputted data from another application (e.g., meals manually logged in My Fitness Pal application, prescription information from an online pharmacy application, etc.). In some embodiments, correlative features may be based on sensor data provided by one or more sensor devices (e.g., wearable sensor devices, smartphone sensor devices, etc.) that report conditions of a user. For example, a wearable sensor device (e.g., a Fitbit device) may provide data about exercise levels of a user. In this example, the data received from the wearable sensor device can be used to determine a correlative feature.

In further reference to FIG. 6, the correlative feature selection UI 600 includes a prompt text element 602 presenting a question related to correlative features. Further, the correlative feature selection UI 600 may also include checkbox elements that enable selecting correlative features. In the depicted example, the correlative feature selection UI 600 incudes a first checkbox element 604 and first text element 614 associated with a food-intake-related correlative feature, a second checkbox element 606 and second text element 616 associated with a medication-intake-related correlative feature, a third checkbox element and third text element 618 associated with a physical-activity-related correlative feature, a fourth checkbox element 610 and fourth text element 620 associated with a stress-level-related correlative feature, and a fifth checkbox element 612 and first text element 622 associated with a sleeping-habit-related correlative feature. In addition, the correlative feature selection UI 600 also includes a button element 624 (e.g., the “Finish” button element) that enables transitioning from the correlative feature selection UI to a subsequent UI such as a home page UI or UIs displaying cross-feature correlation insights, as further described below.

### 2. Block 412: Displaying Cross-Feature Correlation Insights Using Insight UIs

As described above in reference to FIG. 4, at block 412, the process 400 may include displaying cross-feature correlation insights using insight UIs. In some embodiments, the computing device may generate and display an insight flagging UI that enables users to flag a cross-feature correlation insight for user interaction and/or engagement. Further, the computing device may generate and display a cross-temporal insight interaction UI that enables users to select correlation periods and visualize correlation magnitude profiles for flagged cross-feature correlation insights with respect to the user-selected correlation periods. In addition, the computing device may generate and display a set of flagged insight engagement UIs that enable users to edit flagged cross-feature correlation insights and assign metadata fields. Moreover, the computing device may generate and display a set of content engagement UIs that enable users to engage with educational content curated based on the flagged cross-feature insights.

a. Insight Flagging UI

FIG. 7 illustrates an insight flagging UI for displaying (block 412) cross-feature correlation insights as part of the process 400 of FIG. 4, in accordance with certain embodiments of the disclosure. The insight flagging UI 700 is used to display determined cross-feature correlation insights and enable the user to flag the cross-feature correlation insights to interaction and/or engagement (e.g., to discuss in an upcoming medical appointment). As described further above, a cross-feature correlation insight is representative of an inferred correlation between an analyte feature trend for an analyte feature and a correlative feature trend for a correlative feature. Each feature trend describes fluctuations of a feature over time. Accordingly, an analyte feature trend describes fluctuations of a corresponding analyte feature over time, while a correlative feature trend describes fluctuations of a corresponding correlative feature over time. For example, the analyte feature trend for the time-in-range feature describes fluctuations of the time-in-range feature of a user over time. As another example, the correlative feature trend for the sleep correlative feature describes fluctuations of the sleep correlative feature of a user over time. In some embodiments, a cross-feature correlation insight may be associated with a correlation magnitude profile that describes an inferred magnitude of the correlation between an analyte feature trend and a correlative feature trend. For example, the correlation magnitude profile for a cross-feature correlation insight that is associated with a time-in-range feature and a sleep correlative feature may describe that a 20 percent increase in weekly sleep has led to a 10 percent increase in weekly time-in-range.

In reference to FIG. 7, the insight flagging UI 700 includes an add button element 702 (i.e., the “+” button element) that enables connecting additional sensor device(s) and/or additional application(s) to the application 106 of the computing device. The insight flagging UI 700 also includes a navigation element (i.e., the selected “General” navigation element 704) that allows the user to navigate between various UIs as described herein. The insight flagging UI 700 also includes textbox elements that depict notifications for content data other than cross-feature correlation insights. For example, the insight flagging UI 700 includes a first textbox element 706 for activity-related content, a second textbox element 708 for sleep-related content, and a third textbox element 710 for content related to displaying previous events, etc. Further, the insight flagging UI 700 includes an insight interaction element 712 for each cross-feature correlation insight that enables user engagement with the cross-feature correlation insight. As depicted, the insight interaction element 712 for a cross-feature correlation insight depicts an insight textbox element 714 that depicts the correlation magnitude profile for the cross-feature correlation insight, an upper button element 716 (i.e., the “Add to Follow-ups” button element) that enables flagging the depicted cross-feature correlation insight for discussion in a follow-up medical appointment, and a lower button element 718 (i.e., the “LEARN HOW SLEEP AFFECTS TIR” button element) that enables the user to engage with educational content related to the depicted cross-feature correlation insight. In some embodiments, when the user flags a cross-feature correlation insight, the software application adds an indication of the cross-feature correlation insight to the flagged insight engagement UI, as further described below.

b. Cross-Temporal Insight Interaction UI

FIG. 8 illustrates a cross-temporal insight interaction UI for displaying (block 412) cross-feature correlation insights as part of the process 400 of FIG. 4, in accordance with certain embodiments of the disclosure. The cross-temporal insight interaction UI 800 enables the user to select a correlation period for a cross-feature correlation insight and display the correlation magnitude profile of the cross-feature correlation insight in relation to the selected correlation period. In some embodiments, given a cross-feature correlation insight between an analyte feature and a correlative feature, all of the correlation magnitude profiles of the cross-feature correlation insight across all of the available correlation periods are precomputed. Precomputing correlation magnitude profiles increases runtime responsiveness and real-time speed of the cross-temporal insight interaction UI. In some embodiments, the cross-temporal insight interaction UI 800 may be associated with a corresponding one of the one or more cross-feature correlation insights, enable user selection of a selected correlation period of the available correlation periods, and in response to user selection of the selected correlation period, display the correlation magnitude profile for the corresponding cross-feature correlation insight over the selected correlation period.

In reference to FIG. 8, the cross-temporal insight interaction UI 800 includes a navigation element (i.e., the selected “Insights” navigation element 802) that allows the user to navigate between various UIs as described herein. The cross-temporal insight interaction UI 800 also includes a trend line depiction element 804 that depicts a set of fluctuation trend lines for a set of selected features. Further, the cross-temporal insight interaction UI 800 includes a first layer of button elements 806 that enable selecting a correlation period from a set of available correlation periods. In certain embodiments, a default value of the selected correlation period for a depicted cross-feature correlation insight may be determined based on correlation scores associated with the depicted cross-feature correlation insight across a set of correlation periods.

For example, in such embodiments, given a set of A analyte features, B correlative features, and C correlation periods, a computing device determines A*B*C correlation scores. In these embodiments, each correlation score: (i) is associated with a respective one of the analyte features, a respective one of the correlative features, and a respective one of the correlation periods, and (ii) describes a degree of correlation between an analyte feature trend for the respective analyte feature and a correlative feature trend for the respective correlative feature across the respective correlation period. For example, one correlation score may describe the degree of correlation between the time-in-range feature trend and the sleep feature trend across the weekly correlation period.

In some embodiments, given a depicted cross-feature correlation insight that is associated with a respective analyte feature and a respective correlative feature, the default correlation period that is initially selected to display correlation magnitude data of the depicted cross-feature correlation insight may be the period with the highest correlation score among the correlation scores for the respective analyte feature trend and the respective correlative feature trend. In some embodiments, given a depicted cross-feature correlation insight that is associated with a respective analyte feature and a respective correlative feature, the default correlation period that is initially selected to display correlation magnitude data of the depicted cross-feature correlation insight may be the period with the median correlation score among the correlation scores for the respective analyte feature trend and the respective correlative feature trend.

In further reference to FIG. 8, the cross-temporal insight interaction UI 800 also includes a second layer of button elements 808 that enable selecting features whose fluctuation trend lines with respect to the selected correlation period are depicted in the trend line depiction element. In some embodiments, the cross-temporal insight interaction UI 800 may include textbox elements depicted above the trend line depiction element 804 that displays correlation magnitude data associated with the depicted cross-feature correlation insight and the selected correlation period. The textbox elements may be a dynamic element of the cross-temporal insight interaction UI 800 that changes as the user-selected correlation period changes via interaction with the first layer of button elements 806.

c. Flagged Insight Engagement UIs

FIG. 9 illustrates a flagged insight engagement UI for displaying (block 412) cross-feature correlation insights as part of the process 400 of FIG. 4, in accordance with certain embodiments of the disclosure. The flagged insight engagement UI 900 enables the user to view cross-feature correlation insights that were previously flagged by the user, to edit the flagged cross-feature correlation insights, and to assign metadata fields (e.g., future timestamps, such as future appointment dates) to the cross-feature correlation insights. As depicted in FIG. 9, the flagged insight engagement UI 900 includes a navigation element (i.e., the selected “Follow-ups” navigation element 902) that allows the user to navigate between various UIs as described herein. The flagged insight engagement UI 900 an upper button element (i.e., the “Select appointment date” button element 904) that enables assigning a future timestamp (e.g., an appointment date) for the flagged cross-feature correlation insights that are being displayed by the flagged insight engagement UI 900.

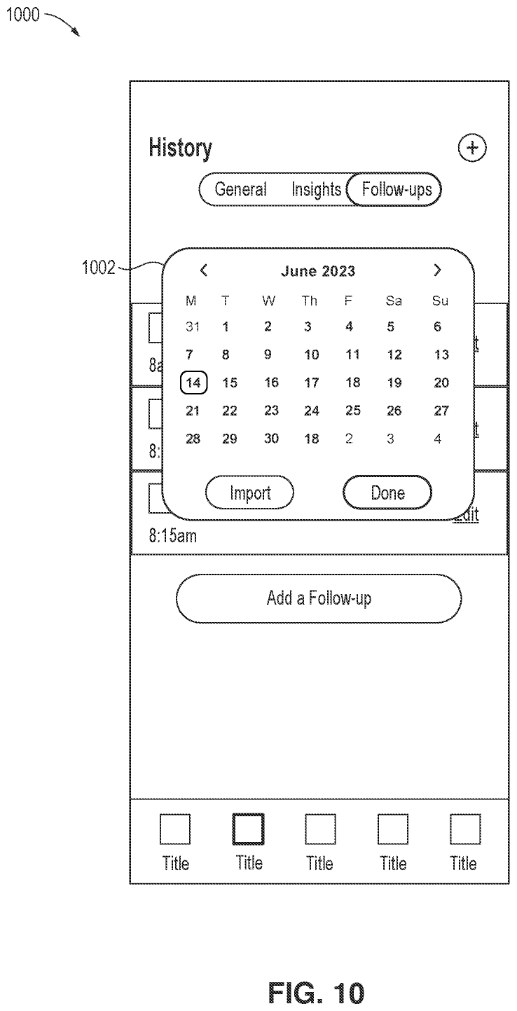

For example, the user selection of the upper button element 904 may cause display of a timestamp selection element (e.g., the date picker element 1002 that is depicted in FIG. 10) to select the future timestamp from a set of available timestamps. Specifically, FIG. 10 illustrates another flagged insight engagement UI for assigning a future timestamp for a flagged cross-feature correlation insight of FIG. 9, in accordance with certain embodiments of the disclosure. The flagged insight engagement UI 1000 may include a date picker element 1002 that allows a user to assign a future timestamp (e.g., an appointment data) for flagged cross-feature correlation insights. In some embodiments, the default value of the future timestamp may be set to a future medical appointment date as indicated by data received from a calendar software application.

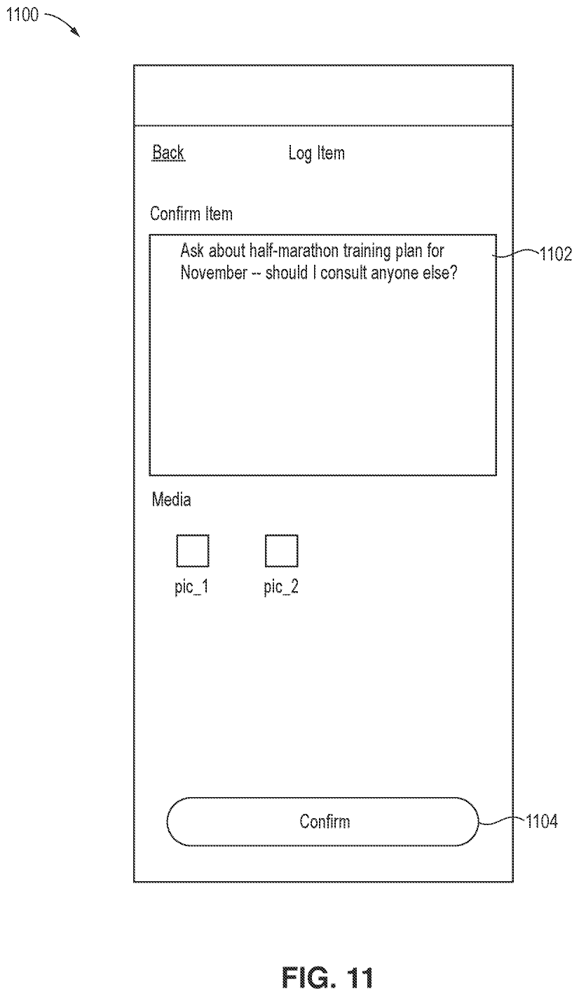

In further reference to FIG. 9, the flagged insight engagement UI 900 also includes a modification button element (i.e., the “Edit” button element) for each depicted cross-feature correlation insight that enables changing description of the cross-feature correlation insight and adding explanatory metadata to the cross-feature correlation insight. For example, the flagged insight engagement UI 900 may include a first cross-feature correlation insight 906 (i.e., “Glucose spiking at night”) and a first modification button element 912, a second cross-feature correlation insight 908 (i.e., “Daily Gym not impacting A1C”) and a second modification button element 914, and a third cross-feature correlation insight 910 (i.e., “10% less sleep this month”) and a third modification button element 916. User selection of the modification button element (e.g., first, second, and third modification buttons 906, 908, 910) for a cross-feature correlation insight may cause display of another flagged insight engagement UI for modifying a flagged cross-feature correlation insight. FIG. 11 illustrates another flagged insight engagement UI for modifying a flagged cross-feature correlation insight of FIG. 9, in accordance with certain embodiments of the disclosure. The flagged insight engagement UI 1100 enable editing metadata fields associated with a selected cross-feature correlation insight and/or adding new metadata fields (e.g., new multimedia content files) for the selected cross-feature correlation insight. For example, the flagged insight engagement UI 1100 may include a text element 1102 that describes an item related to a flagged cross-feature correlation insight and a confirmation element 1104 that enables the user to confirm the information in the text element 1102.

In further reference to FIG. 9, the flagged insight engagement UI 900 includes an add button element 920 (i.e., the “+” button element) that enables adding new items (e.g., other non-flagged cross-feature correlation insights, user-entered follow-up items, etc.) to the flagged insight engagement UI. Further, the flagged insight engagement UI 900 also includes a lower button element 918 (i.e., the “Add to Follow-up” button element) that enables flagging the depicted cross-feature correlation insight for discussion in a follow-up medical appointment.

d. Content Engagement UIs

The content engagement UIs enable user engagement with educational content curated for the user. In various embodiments, the content engagement UIs enable displaying educational content that is curated based on determined cross-feature correlation insights for a user, based on analyte features and correlative features selected by the user, and/or based on any other user input (e.g., application objectives selected by the user).

FIG. 12 illustrates a content engagement UI for displaying cross-feature correlation insights determined as part of the process 400 of FIG. 4, in accordance with certain embodiments of the disclosure. The content engagement UI 1200 includes an educational content text element 1202 that is a title of education content (i.e., “How Sleep Habits Impact Glucose Levels”). The user may engage with the education content using a start element 1204 and the user may rate the particular education content using a rating element 1206 (e.g., stars from 1 to 5).

FIG. 13 illustrates another content engagement UI for engaging with education content of FIG. 12, in accordance with certain embodiments of the disclosure. Upon the user selecting the start element 1204, the user may be presented with the content engagement UI 1300 that includes education content text element 1302. In addition, the user may be presented with an engagement prompt element 1304 (i.e., “How many hours of sleep do you aim to achieve each night?”) and the user may provide answer using the first checkbox element 1306 and first text element 1314 (i.e., “0-4 hours”), second checkbox element 1308 and second text element 1316 (i.e., “4-6 hours”), third checkbox element 1310 and third text element 1318 (i.e., “6-8 hours”), and fourth checkbox element 1312 and fourth text element 1320 (i.e., “8+ hours”). In some embodiments, the content engagement UI 1300 may include a next element 1322 that enables the user to see and engage with more education content.

### Example Apparatus for Dynamic Determination and Presentation of Cross-Feature Correlation Insights

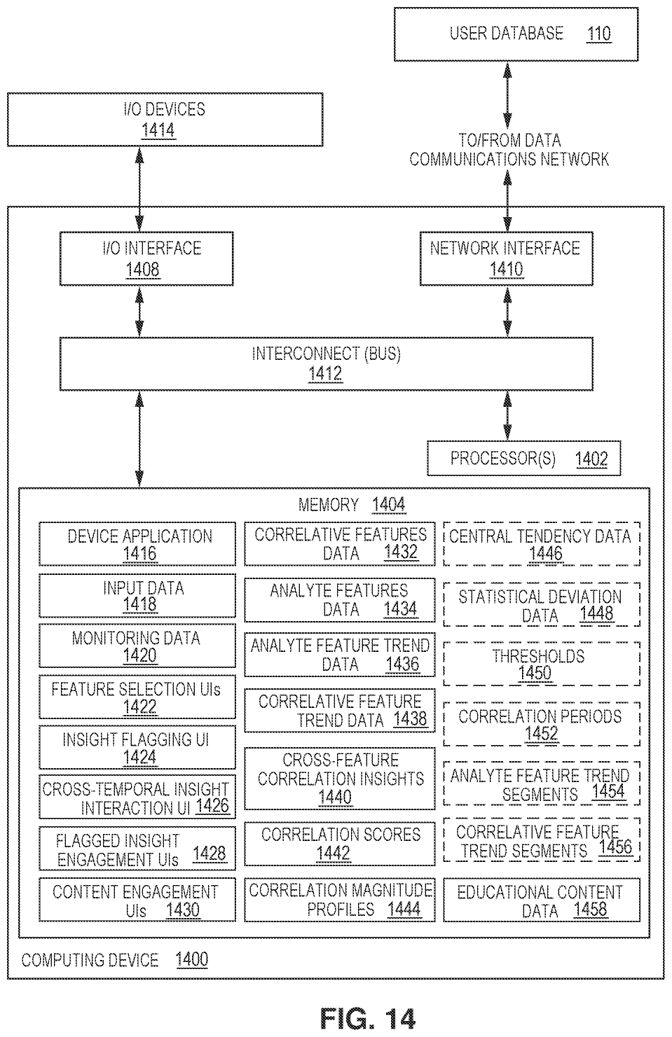

FIG. 14 is a block diagram depicting a computing device 1400 configured for dynamic determination and presentation of cross-feature correlation insights, in accordance with certain embodiments disclosed herein. Although depicted as a single physical device, in embodiments, computing device 1400 may be implemented using virtual device(s), and/or across a number of devices, such as in a cloud environment. Computing device 1400 may be mobile device 107, a server, multiple serves, or any combination thereof.

As illustrated, computing device 1400 includes a one or more processor(s) 1402, memory 1404 (e.g., volatile memory or non-volatile memory), a network interface 1410, and one or more input/output (I/O) interfaces 1408. In the illustrated embodiment, processor 1402 retrieves and executes programming instructions stored in the memory 1404, as well as stores and retrieves data residing in the memory 1404. In certain embodiments, memory 1404 is configured to store instructions (e.g., computer-executable code, device application 1416) that when executed by processor(s) 1402, cause processor(s) 1402 to perform the processes and/or operations described herein and illustrated in FIGS. 3-13. In certain embodiments, memory 1404 stores code for executing the functions of the DAM 111, decision support engine 112, and/or the application 106. Note that computing device 1400 may be configured to perform the functions of only one of the DAM 111, decision support engine 112, and/or the application 106, in which case additional system(s) may be used for performing the functions of the others.

Processor(s) 1402 is generally representative of a single central processing unit (CPU) and/or graphics processing unit (GPU), multiple CPUs and/or GPUs, a single CPU and/or GPU having multiple processing cores, and the like. In certain embodiments, memory 1404 may be a volatile memory, non-volatile memory, or combination of volatile and non-volatile memories. For example, volatile memory may include a random access memory (RAM). Non-volatile memory may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1414 (such as keyboards, monitors, etc.) can be connected via the I/O interface(s) 1408. Further, via network interface 1410, computing device 1400 can be communicatively coupled with one or more other devices and components, such as user database 110. In certain embodiments, computing device 1400 is communicatively coupled with other devices via a network, which may include the Internet, local network(s), and the like. The network may include wired connections, wireless connections, or a combination of wired and wireless connections. As illustrated, processor(s) 1402, memory 1404, network interface 1410, and I/O interface(s) 1408 are communicatively coupled by one or more bus interconnects 1412. In certain embodiments, computing device 1400 is a server executing in an on-premises data center or a cloud environment. In certain embodiments, the computing device 1400 is a user's mobile device.

In the illustrated embodiment, the memory 1404 may include a device application 1416 that configures the processor(s) 1402 to perform various processes and/or operations in dynamic determination and presentation of cross-feature correlation insights, as described above. In some embodiments, the device application 1416 may perform the functions of the DAM 111, the decision support engine 112, and/or the application 106. As described above in reference to FIGS. 3-13, the computing device 1400 may be configured to identify analyte features and correlative features using feature selection UIs 1422 such as, but not limited to, an analyte feature selection UI and a correlative feature selection UI. In some embodiments, the analyte features and/or the correlative features may be identified based on various data such as, but not limited to, input data 1418 (e.g., inputs 127, etc.), as further described above. In some embodiments, the analyte features and related data may be stored as analyte features data 1434. In some embodiments, the correlative features and related data may be stored as correlative features data 1432. In addition, the computing device 1400 may be configured to determine analyte feature trends and correlative feature trends based on various data such as, but not limited to, monitoring data 1420 (e.g., metrics 130, etc.), as further described above. In some embodiments, the analyte feature trends and related data may be stored as analyte feature trend data 1436. In some embodiments, the correlative feature trends and related data may be stored as correlative feature trend data 1438.

In further reference to the illustrated embodiment, computing device 1400 may be configured to determine cross-feature correlation insights 1440 and correlation magnitude profiles 1444, as further described above. For example, the computing device 1400 may be configured to determine cross-feature correlation insights 1440 and/or correlation magnitude profiles 1444 using correlation scores 1442, thresholds 1450, correlation periods 1452, central tendency data 1446, statistical deviation data 1448, analyte feature trend segments 1454, and correlative features trend segments 1456. Furthermore, the computing device 1400 may be configured to generate and display cross-feature correlation insights 1432 using insight UIs such as, but not limited to, an insight flagging UI 1424, cross-temporal insight interaction UI 1426, flagged insight engagement UIs 1428, and content engagement UIs 1430, as further described above. In some embodiments, the computing device 1400 may be configured store education content data 1458, which may be used to display curated education content using the content engagement UIs 1430, as further described above. In some embodiments, the correlation magnitude profiles 1444 may be stored in a high-speed storage medium (e.g., a volatile memory) to further enhance real-time responsiveness of the cross-temporal insight interaction UIs 1426.

Further, although specific operations (e.g., operations of FIGS. 3-13) and data are described as being performed and/or stored by a specific computing device above with respect to FIG. 14, in certain embodiments, a combination of computing devices may be utilized instead.

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” In this document, the term “set” or “a set of” a particular item is used to refer to one or more than one of the particular item.

Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel,” “perpendicular,” “round,” or “square,” are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A non-transitory computer readable storage medium storing a program comprising instructions that, when executed by at least one processor of a computing device, cause the at least one processor to perform operations including:
identifying at least one analyte feature using an analyte feature selection user interface (UI);
identifying at least one correlative feature using a correlative feature selection UI;
determining an analyte feature trend for the at least one analyte feature, wherein the analyte feature trend corresponds to fluctuations of the at least one analyte feature over a period of time;
determining a correlative feature trend for the at least one correlative feature, wherein the correlative feature trend corresponds to fluctuations of the at least one correlative feature over the period of time;
determining at least one cross-feature correlation insight based on the analyte feature trend and the correlative feature trend, wherein the at least one cross-feature correlation insight is based on a correlation between the at least one analyte feature and the at least one correlative feature;
determining a correlation magnitude profile for the at least one cross-feature correlation, wherein the correlation magnitude profile includes correlation magnitudes for the at least one cross-feature correlation insight over a correlation period; and
displaying the at least one cross-feature correlation insight using at least one insight UI.

2. The non-transitory computer readable storage medium of claim 1, wherein the at least one analyte feature is identified by displaying the analyte feature selection UI and receiving a user input selecting the at least one analyte feature.

3. The non-transitory computer readable storage medium of claim 2, wherein the at least one corresponding feature is identified by displaying the corresponding feature selection UI and receiving a user input selecting the at least one corresponding feature.

4. The non-transitory computer readable storage medium of claim 1, wherein the at least one cross-feature correlation insight is displayed using an insight flagging UI, and wherein the insight flagging UI provides UI elements for a user to flag a cross-feature correlation insight.

5. The non-transitory computer readable storage medium of claim 4, wherein the at least one cross-feature correlation insight is displayed using a cross-temporal insight interaction UI, and wherein the cross-temporal insight interaction UI:
provides UI elements for the user to select correlation periods; and
displays the correlation magnitude profile for the flagged cross-feature correlation insight with respect to the user-selected correlation periods.

6. The non-transitory computer readable storage medium of claim 5, wherein the operations further comprise:
receiving a user selected correlation period using the cross-temporal insight interaction UI;
updating the correlation magnitude for the flagged cross-feature correction insight; and
displaying the updated correlation magnitude profile for the flagged cross-feature correlation insight using the interaction UI.

7. The non-transitory computer readable storage medium of claim 5, wherein the at least one cross-feature correlation insight is displayed using a set of flagged insight engagement UIs, and wherein the set of insight engagement UIs:
provide UI elements for the user to edit the flagged cross-feature correlation insight; and
assign metadata fields to the flagged cross-feature correlation insight.

8. The non-transitory computer readable storage medium of claim 7, wherein the at least one cross-feature correlation insight is displayed using a set of content engagement UIs, and wherein the set of content engagement UIs:
display educational content curated based on the flagged cross-feature insight; and
provide UI elements for the user to engage with the educational content.

9. A method for dynamic determination and presentation of cross-feature correlation insights, the method comprising:
identifying at least one analyte feature using an analyte feature selection user interface (UI);
identifying at least one correlative feature using a correlative feature selection UI;
determining an analyte feature trend for the at least one analyte feature, wherein the analyte feature trend corresponds to fluctuations of the at least one analyte feature over a period of time;
determining a correlative feature trend for the at least one correlative feature, wherein the correlative feature trend corresponds to fluctuations of the at least one correlative feature over the period of time;
determining at least one cross-feature correlation insight based on the analyte feature trend and the correlative feature trend, wherein the at least one cross-feature correlation insight is based on a correlation between the at least one analyte feature and the at least one correlative feature;
determining a correlation magnitude profile for the at least one cross-feature correlation, wherein the correlation magnitude profile includes correlation magnitudes for the at least one cross-feature correlation insight over a correlation period; and
displaying the at least one cross-feature correlation insight using at least one insight UI.

10. The method of claim 9, wherein the at least one analyte feature is identified by displaying the analyte feature selection UI and receiving a user input selecting the at least one analyte feature.

11. The method of claim 10, wherein the at least one corresponding feature is identified by displaying the corresponding feature selection UI and receiving a user input selecting the at least one corresponding feature.

12. The method of claim 9, wherein the at least one cross-feature correlation insight is displayed using an insight flagging UI, and wherein the insight flagging UI provides UI elements for a user to flag a cross-feature correlation insight.

13. The method of claim 12, wherein the at least one cross-feature correlation insight is displayed using a cross-temporal insight interaction UI, and wherein the cross-temporal insight interaction UI:
provides UI elements for the user to select correlation periods; and
displays the correlation magnitude profile for the flagged cross-feature correlation insight with respect to the user-selected correlation periods.

14. The method of claim 13, wherein the at least one cross-feature correlation insight is displayed using a set of flagged insight engagement UIs, and wherein the set of insight engagement UIs:
provide UI elements for the user to edit the flagged cross-feature correlation insight; and
assign metadata fields to the flagged cross-feature correlation insight.

15. The method of claim 14, wherein the at least one cross-feature correlation insight is displayed using a set of content engagement UIs, and wherein the set of content engagement UIs:
display educational content curated based on the flagged cross-feature insight; and
provide UI elements for the user to engage with the educational content.

16. A computing device for dynamic determination and presentation of cross-feature correlation insights, the computing device comprising:
a network interface;
a memory comprising executable instructions;
a processor in data communication with the memory and configured to execute the instructions to:
identify at least one analyte feature using an analyte feature selection user interface (UI);
identify at least one correlative feature using a correlative feature selection UI;
determine an analyte feature trend for the at least one analyte feature, wherein the analyte feature trend corresponds to fluctuations of the at least one analyte feature over a period of time;
determine a correlative feature trend for the at least one correlative feature, wherein the correlative feature trend corresponds to fluctuations of the at least one correlative feature over the period of time;
determine at least one cross-feature correlation insight based on the analyte feature trend and the correlative feature trend, wherein the at least one cross-feature correlation insight is based on a correlation between the at least one analyte feature and the at least one correlative feature;
determine a correlation magnitude profile for the at least one cross-feature correlation, wherein the correlation magnitude profile includes correlation magnitudes for the at least one cross-feature correlation insight over a correlation period; and
display the at least one cross-feature correlation insight using at least one insight UI.

17. The computing device of claim 16, wherein the at least one analyte feature is identified by displaying the analyte feature selection UI and receiving a user input selecting the at least one analyte feature.

18. The computing device of claim 17 wherein the at least one corresponding feature is identified by displaying the corresponding feature selection UI and receiving a user input selecting the at least one corresponding feature.

19. The computing device of claim 16, wherein the at least one cross-feature correlation insight is displayed using an insight flagging UI, and wherein the insight flagging UI provides UI elements for a user to flag a cross-feature correlation insight.

20. The computing device of claim 17, wherein the at least one cross-feature correlation insight is displayed using a cross-temporal insight interaction UI, and wherein the cross-temporal insight interaction UI:
provides UI elements for the user to select correlation periods; and
displays the correlation magnitude profile for the flagged cross-feature correlation insight with respect to the user-selected correlation periods.
